(19) United States
(12) Patent Application Publication
(10) Pub. No.: US 2026/0297481 A1
(43) Pub. Date: Oct. 1, 2026

(54) BIO-ENGINEERED HUMAN PROSTATE GLAND AND DISEASE MODEL

(71) Applicant: ARIZONA BOARD OF REGENTS ON BEHALF OF THE UNIVERSITY OF ARIZONA, Tucson, AZ (US)

(72) Inventors: Linan JIANG, Tucson, AZ (US); Cynthia MIRANTI, Tucson, AZ (US)

(21) Appl. No.: 19/489,902

(22) PCT Filed: Jun. 7, 2024
(86) PCT No.: PCT/US2024/032983

§ 371 Date: Dec. 4, 2025

Related U.S. Application Data
(60) Provisional application No. 63/506,691, filed on Jun. 7, 2023.

(51) Int. Cl. C12M 3/00; C12M 1/00; C12M 3/06

(52) U.S. Cl. CPC C12M 21/08; C12M 23/16; C12M 23/40; C12M 29/04

(57) ABSTRACT

Microfluidic devices, organ-on-a-chip platforms (e.g. mimicking the human prostate gland), and uses thereof are described herein. The microfluidic device includes at least two layers and a porous membrane therebetween. Each layer includes a channel, and the first channel and the second channel are aligned such that a first region of a first channel overlaps with a region of the second channel, and a second region of the first channel does not overlap a second region of the second channel. A disease model using the microfluidic devices includes (i) placing a first type of cells in the first channel and (ii) flowing a first cell culture medium through the first channel. Optionally, in step (i) a second type of cells in the second channel and in step (ii) a second cell culture medium flows through the second channel. The microfluidic devices can be used in drug screening and testing.

### CROSS-REFERENCE TO RELATED APPLICATION

This application claims the benefit of and priority to U.S. Provisional Application No. 63/506,691 filed Jun. 7, 2023, the entire content of which is incorporated herein by reference for all purpose in its entirety.

### STATEMENT REGARDING FEDERALLY SPONSORED RESEARCH OR DEVELOPMENT

This invention was made with government support under CA254200 awarded by the National Institutes of Health. The government has certain rights in the invention.

### FIELD OF THE INVENTION

The disclosed invention is generally in the field of microfluidic devices.

### BACKGROUND OF THE INVENTION

There is need for alternatives to animal studies for understanding the development of and progression of diseases, such as cancer, and to assist in the development of pharmaceuticals and determine appropriate dosing regimens.

Model systems that recapitulate the functional features observed in tissues are of high demand to study morphogenesis as well as disease initiation and progression. However, insight into the underlying biological mechanisms remains limited. One of the major hurdles is the gap between the mechanism-defining in vitro single-type monolayered cell culture (MCC) models and the physiology-relevant in vivo animal model. Current MCC models, although being extremely useful in delineating intracellular mechanics, have proven to be a poor predictor of physiological responses. The lack of critical communications between different cell types in the MCC models has been the major reason for this failure. Signaling pathways between different cell types have long been implicated in morphogenesis and disease development.

Microfluidic systems have already enabled various biological studies. Multicompartmental 3D microfluidic cell culture devices, often termed as organs-on-chips (OoC), have been introduced to address limitations of in vitro modeling. So far, numerous OoC devices have been proposed to reproduce various functions of organs and tissues, such as lung, liver, kidney, intestine, gut, and bone. However, despite the high prevalence of life-threatening diseases and cancers that affect exocrine glands, an organ-on-a-chip system for in vitro modeling of a human prostate gland is yet to be developed.

Therefore, a model system that recreates the architectural features to recapitulate the biology and physiology of the human prostate gland in vivo is needed.

### SUMMARY OF THE INVENTION

Microfluidic devices and organ-on-a-chip platforms based on microfluidic devices are described herein. For example, an in vitro human prostate disease model based on the disclosed microfluidic devices is described in the Examples. The microfluidic devices include multiple individual compartments separated by porous membranes. Co-cultures of tissue types involved in prostate diseases are implemented while providing physiological environment and communications among the tissue types.

The microfluidic device includes multiple individual compartments separated by porous membranes (see, e.g., FIG. 2b). The microfluidic device includes at least two layers and at least one porous membrane that is adjacent to each of and between two neighboring layers. The two adjacent layers of the microfluidic device can be arranged in any suitable positions, such as in parallel with each other.

The device may contain more than two layers where each layer is separated from its adjacent layer by a porous membrane. The two layers (when the microfluidic device includes two layers) or at least two layers (when the microfluidic device includes more than two layers) of the microfluidic device each include one or more channels, such as one channel or two or more channels.

The microfluidic device includes a feed area (see, e.g., FIG. 3a, 1110), an exit area (see, e.g., FIG. 3a, 1120), and a material exchange area (see, e.g., FIG. 3a, 1130). Within the material exchange area, the channel or each channel (when two or more channels are present) in a layer is aligned with the channel or each channel (when two or more channels are present) in its neighboring layer or each neighboring layers, such that a region of the channel or each channel in the layer overlaps with a region of the channel or each channel in its neighboring layer or each neighboring layer, and a region of the channel or each channel in the layer does not overlap with any region of the channel or each channel in its neighboring layer or each neighboring layer. For example, within the material exchange area of a microfluidic device, a first channel in a first layer is aligned with a second channel in a second, adjacent layer, such that a first region (see, e.g., FIG. 1b, 1040a) of the first channel in the first layer overlaps with a second region (see, e.g., 1050a not visible in FIG. 1b) of the second channel in the second layer, and a third region (see, e.g., FIG. 1b, 1060a) of the first channel does not overlap with any region of the second channel in the material exchange area.

Within the material exchange area of the microfluidic device, the channel or each channel in a layer may include more than one region that overlaps with the channel or each channel in its neighboring layer or each neighboring layer, such as two or more overlapping regions, for example, two, three, four, or five, or more than five overlapping regions (see, e.g., FIG. 1a, 1040a, 1040b, 1040c, 1040d, 1040e, 1040f, and 1040g); and/or more than one region that does not overlap with any region in its neighboring layer or each neighboring layer, such as two or more non-overlapping regions, for example, two, three, four, or five, or more than five non-overlapping regions (see, e.g., FIG. 1a, 1060a, 1060b, 1060c, 1060d, 1060e, 1060f, and 1060g). In some forms, the microfluidic device can include one or more reservoirs in fluid connection with one or more channels in at least one layer of the microfluidic device, optionally positioned at the overlapping region(s) of the channel(s).

The geometry of the channel or each channel in each layer can have any suitable shape, as long as it can be aligned with a channel in the neighboring layer such that one or more overlapping region(s) and one or more non-overlapping regions are achieved with the channel in the neighboring layer, within the material exchange area of the microfluidic device. Suitable shapes for each channel in the microfluidic device include, but are not limited to, straight and meander, such as a bent, curved, or serpentine shape. For example, each channel in the microfluidic device is a straight or meandering channel, such as a channel having a bent, curved, or serpentine configuration.

The shape of the channel or each channel in a layer may be the same as or different from the channel or each channel in its neighboring layer. For example, the channel or each channel in a first layer of a microfluidic device may be different from the channel or each channel in a second layer. For example, a first channel in the first layer of a microfluidic device can be substantially straight, while a second channel in the second layer is a meandering channel, such as a channel having a bent, curved, or serpentine configuration; or vice versa. For example, a first channel in the first layer is a meandering channel, such as a channel having a bent, curved, or serpentine configuration, while each of a second and third channel in the second layer can be substantially straight. For example, a first channel in the first layer can be substantially straight, which each of a second and third channels is a meandering channel, such as a channel having a bent, curved, or serpentine configuration. For example, a first channel in the first layer of a microfluidic device is meandering channel, such as a channel having a bent, curved, or serpentine configuration; a second channel in the second layer can be substantially straight, and a third channel in the third layer is a meandering channel, such as a channel having a bent, curved, or serpentine configuration.

The pore sizes and/or porosities of the membranes between different layers are selected based on the types of cells in the channel(s) to permit diffusion of substances secreted by the cells between the adjacent channels, or to allow cell invasion through the pores to the adjacent channels, or a combination thereof. Typically, the substances secreted by the cells are soluble in water or an aqueous solution, such as an aqueous buffer. Examples of secreted substances by cells include, but are not limited to PSA, TGFβ, PDGF, TNFα, exosomes, Wnts, cytokines, FGF, KGF, and HGF. In a device containing two or more porous membranes, the pore sizes and porosities of each porous membrane can be the same or different, optionally they are different.

A disease model can be constructed using the microfluidic devices described herein by (i) placing a first type of cells in a first channel of the first layer, and (ii) flowing a first cell culture medium through the first channel. The first type of cells may be placed at any location within the first channel. For example, the first type of cells is placed on a bottom surface of the porous membrane that functions as the ceiling of the channel in the first layer (see, e.g., FIG. 3c, normal endothelial cells placed on bottom surface 313 of the porous membrane 310). Optionally, in step (i) a second type of cells is placed in the first channel and/or a second channel in the second layer, and in step (ii) a second cell culture medium flows through the first and/or second channel. Alternatively, no cells are placed in the second channel in a second layer of the microfluidic device. Optionally, when the microfluidic device includes a third channel in the first or second, or a third layer, in step (i) a third type of cells is placed in the third channel, and in step (ii) flowing a third cell culture medium through the third channel. Alternatively, although the microfluidic device includes a third channel in the first, second, or third layer, no cells are placed in the third channel. In some forms, more than one type of cells and/or tissues can be placed in a single channel. For example, in step (i), at least two types of cells are placed in the first channel of the first layer. The cells being placed in the channel(s) of the microfluidic device can be in any suitable form. For example, the cells can be placed in the channel(s) of the microfluidic device in either 2D form (for example, in the form of a monolayer) or a 3D form (for example, in the form of organoids, spheroids, one or more types of cells embedded in hydrogel or ex vivo tissue, etc.). Each channel in the microfluidic device may contain a different type of cells or the same type of cells, or a combination thereof.

The microfluidic devices described herein can be used in drug screening. In some embodiments, steps (i) and (ii) as described above are followed along with (iii) flowing a drug solution or suspension, or flowing a medium containing one or more carriers containing one or more drugs (also referred to herein as “drug carrier medium”), through the first channel and/or the second channel. The drug carrier medium contains a suitable medium and one or more drug carriers that contain a suitable concentration of drug(s), such as a high concentration of drugs, where the drugs can be enclosed/embedded in the carriers, such as small-volume capsules and/or beads. The drug carrier can be formed by any suitable materials, such as gelatin, collagen, Matrigel, agarose, alginate, etc.

When a drug carrier medium is used in the method, the method may further include after step (iii), flowing a cell culture medium (without any drugs) through the first channel and/or the second channel.

When the microfluidic device includes more than two layers and/or more than two channels in each layer, in step (iii) a different or the same drug solution or suspension, or drug carrier medium, can be flown through the channel(s) of each layer. A different drug solution or suspension refers to a solution or suspension that contains a different drug or different combination of drugs, a different drug concentration, etc. The same drug solution or suspension refers to a solution or suspension that contains the same drug(s), each drug having the same concentration. A different drug carrier medium refers to a medium that contains a different carrier, a different carrier concentration, a different drug or combination of drugs, a different drug loading, etc. The same drug carrier medium refers to a medium that contains the same carrier, same drug(s), same carrier concentration, and same drug loading.

For example, in a microfluidic device including two layers, each layer containing two or more channels, a first drug solution/suspension or a first drug carrier medium flows through the channels of the first layer, and a second, different drug solution/suspension or drug carrier medium flows through the channels of the second layer. Alternatively, for example, in a microfluidic device including two layers, each layer containing two or more channels, a drug solution/suspension or drug carrier medium flows through the channels of the first layer, and the same drug solution/suspension or drug carrier medium flows through the channels of the second layer. In these forms, however, the dosing regime, time, frequency, etc. for flowing the drug solution/suspension or drug carrier medium in each layer may be the same or different. For example, the drug solution/suspension or carrier medium flows through the channels of the first layer at a different dosing regime, time, and/or frequency compared to the drug solution/suspension or drug carrier medium flowing through the channels of the second layer.

The devices described herein can be used to form an organ-on-a chip platform, such as one which mimics the prostate gland and/or portions of the tissues therein. Optionally, the prostate gland on a chip can be in fluid communication, such as via flow convection or diffusion, with one or more additional and different organs on a chip to study the interactions between different organs, optionally during the development of and/or possible treatments of diseases therein.

### BRIEF DESCRIPTION OF THE DRAWINGS

FIGS. 1a-1c show an exemplary microfluidic device containing two flow channels with selectively overlapping regimes separated by a porous membrane. FIG. 1a shows a top view of the microfluidic device, illustrating the alignment of the two channels. FIG. 1b shows a zoom-in view of the device shown in FIG. 1a. FIG. 1c shows a cross-sectional view at location (A-A) of the device shown in FIG. 1a.

FIG. 2a shows the fabrication of a PDMS microchannel replicated from mater molds for an exemplary microfluidic device. FIG. 2b shows an exploded view of the exemplary microfluidic device.

FIG. 3a shows an exemplary microfluidic device for a human prostate disease model. FIG. 3b shows an A-A cross-sectional view of the microfluidic device of FIG. 3a, where a vasculature system including normal endothelial cells (such as in the form of a monolayer) and immune cells are placed in the first channel; stroma including fibroblast and smooth muscle cells (such as in the form of stroma embedded in 3D ECM hydrogel) are placed in the second channel; and normal prostate epithelial cells or cancer cells (such as in the form of a monolayer or organoids or in 3D Matrigel, or a combination thereof). FIG. 3c shows that in this exemplary human prostate disease model, stroma were converted to CAFs by cancer cells; cancer cells invaded into stroma and further invaded into bloodstream; and immune cells responded to the invasion by cancer cells. These models can be used in drug treatment tests, where a drug solution or suspension can flow through either one of the channels or a combination thereof.

FIG. 4a shows an exemplary microfluidic device for cancer in situ model. FIG. 4b shows an A-A cross-sectional view of the microfluidic device of FIG. 4a, where stroma including fibroblast and smooth muscle cells are placed in the first channel; epithelium and locally induced tumor are placed in the second channel; and the third channel is for administering treatment, such as Dox, IPTG, or DHT, or a combination thereof. FIG. 4c shows that the treatment induced local tumor in epithelium; tumor cells converted normal stroma to CAFs, and tumor invasion, following the cell placement and drug treatment as shown in FIG. 4b.

FIG. 5a shows an exemplary microfluidic device for modeling the effect of tumor on healthy/normal epithelium. FIG. 5b shows an A-A cross-sectional view of the microfluidic device of FIG. 5a, where stroma including fibroblast and smooth muscle cells are placed in the first channel; cancer cells are placed in the second channel; and normal epithelium are placed in the third channel. FIG. 5c shows the co-culture of cancer cells with normal epithelium and stroma, operate under conditions (e.g. flow rate) and selected membrane pore size to allow diffusion of cancer cell secretion (0.8 um or 8 um pore membrane) and invasion of cancer cells (8 um pore membrane), following the cell placement as shown in FIG. 5b. This exemplary model can be used to study the interaction between tumor cells and micro-environment, stroma conversion to CAF by cancer cells with or without physical contact, the effect of tumor cells and surrounding normal epithelium cells, and/or changes in normal epithelium.

FIG. 6a shows an exemplary system including two connected exemplary microfluidic devices for modeling prostate tumor metastasis to a secondary organ (for example a bone chip). FIG. 6b shows an A-A cross-sectional view of the prostate chip and a B-B cross-sectional view of the bone chip in the system of FIG. 6a.

FIG. 7a shows an exemplary microfluidic device containing channel arrays for a high-throughput drug screening disease model, where two sets of channel arrays are separated by a porous membrane, with overlapping alignment regimes between the upper channel array and the lower channel array, and a uniform depth for the upper and lower channels. FIG. 7b shows an A-A cross-sectional view of the microfluidic device of FIG. 7a. FIG. 7c shows a B-B cross-sectional view of the microfluidic devices in the system of FIG. 7a. This exemplary device can be used for high-throughput drug screening disease model targeting tumor/stroma/stroma-tumor interaction, such as by injecting culture medium with or without drug into the lower and/or upper channel arrays; delivering the drug into upper or lower channel array to specifically target tumor, or stroma, or stroma-tumor interaction or delivering a combination of drugs into one of or both channel arrays; and scheduling drug concentration, dose, time, frequency, etc. for each channel array or for individual channels in each array.

FIG. 8a shows another exemplary microfluidic device containing channel arrays for a high-throughput drug-screening disease model, where two sets of channel arrays are separated by a porous membrane, with overlapping alignment regimes between the upper channel array and the lower channel array. The upper channels have uniform depth, and the lower channels contain wells at the overlapping regimes. FIG. 8b shows an A-A cross-sectional view of the microfluidic device of FIG. 8a. FIG. 8c shows a B-B cross-sectional view of the microfluidic devices in the system of FIG. 8a. This exemplary device can be used for high-throughput drug screening disease model targeting tumor/stroma/stroma-tumor interaction, such as by placing tumor cells (or stroma) on the membrane surface in the lower channels; placing stroma (or tumor cells) on the membrane surface in the upper channels; injecting suspension of drug-containing capsules/particles in culture medium in the lower channels, such that the drug capsules/particles are collected in the wells, where the drug-containing capsules/particles dissolve in culture medium in a time-dependent manner to release drug locally in the wells; and varying medium flows in the top and/or bottom channels to result in various local drug concentration in the overlapping regimes.

FIGS. 9a-9d show an exemplary disease model by placing stroma and tumor cells in separate channels of an exemplary microfluidic device with no initial direct physical contact. FIG. 9a shows the exemplary microfluidic device where tumor cells and normal stroma are placed in separate channels on the porous membrane. FIG. 9b shows a zoom-in view of the channels of the microfluidic device of FIG. 9a. FIG. 9c shows invasion of tumor cells into stroma, and stroma conversion into CAFs in the tumor microenvironment, following the cell placement as shown in FIG. 9b. FIG. 9d shows a zoom-in view of the channels of the microfluidic device of FIG. 9c.

FIGS. 10a-10d show another exemplary disease model by placing stroma and tumor cells in the channel of an exemplary microfluidic device with direct physical contact. FIG. 10a shows the exemplary microfluidic device where tumor cells are placed on a monolayer of stroma, with direct physical contact. FIG. 10b shows a zoom-in view of the channels of the microfluidic device of FIG. 10a. FIG. 10c shows enhanced invasion of tumor cells into neighboring space facilitated by invading CAFs converted by tumor cells, following the cell placement as shown in FIG. 10b. FIG. 10d shows a zoom-in view of the channels of the microfluidic device of FIG. 10c.

FIGS. 11a-11c show simulations of the convection-diffusion flow in an exemplary microfluidic device. FIG. 11a is a schematic illustrating a 2D physical model used for simulations depicting transport of signaling molecules through the porous membrane due to diffusion from the secreting layer to the receiving layer channel. The gradient map in the two channels illustrates concentration changes from high (black) to low (white). FIG. 11b is a graph showing the 2D simulation result of streamwise normalized concentration profiles of secreted molecules along both channels under various flowrates, at a distance 40 μm away from membrane, with the corresponding rainbow maps of planar concentration distributions. FIG. 11c is a graph showing the simulation result of cross-stream normalized concentration profiles of secreted molecules, at mid-distance between channel inlet and outlet, under various flowrates. All results were obtained from a device with 8 μm-pore membrane, except curves in dotted lines in FIG. 11b and FIG. 11c, which were obtained from a device with 0.8 um-pore membrane at a flow rate of 30 μl/hr.

FIG. 12 is a graph showing migration rate of invading cancer cells for various initial cell plating conditions based on measured time-dependent distance between the edge of original plating channel of cancer cells and the farthest cancer cell migration front in the neighboring channel. The filled symbols (22Rv1/BHP or C4-2/BHP) indicate co-culture of cancer cells 22Rv1 or C4-2 with stromal BHPrS1 fibroblasts on opposite surfaces of the separation membrane as shown in the exemplary disease model of FIGS. 9a-9d. Empty symbols (22Rv1-on-BHP or C4-2-on-BHP) indicate co-culture of cancer cells 22Rv1 or C4-2 cancer cells with stroma BHPrS1 fibroblast while the cancer cells were placed on a monolayer of fibroblasts in the same channel as shown in the exemplary disease model of FIGS. 10a-10d. Values in distance were represented as average ±SD of 4 replicates.

FIGS. 13a-13c are graphs showing cytokine production in a prostate cancer-on-chip model as shown in the exemplary disease model of FIGS. 10a-10d. FIG. 13a shows the levels of TGFβ in the straight channel. FIG. 13b shows the levels of TGFβ in the serpentine channel. FIG. 13c shows the levels of PDGF in the serpentine channel. The data were measured in conditioned medium by ELISA. PDGF was below detectable levels (not shown) in the medium collected from straight channels of both experiments. The conditioned medium was collected every two days starting at Day 7, from straight and serpentine channels of 8 μm-pore devices. The straight channel contained BHPrS1 cells and tumor cells, while the serpentine channel contained invaded stromal fibroblasts and tumor cells. Error bars=SD; n=4; *<0.05; **<0.001 relative to Day 7 values.

### DETAILED DESCRIPTION OF THE INVENTION

### I. Microfluidic Devices

Microfluidic devices and organ-on-a-chip platforms based on these microfluidic devices are described herein. For example, an in vitro human prostate disease model based on the disclosed microfluidic devices is described in the Examples.

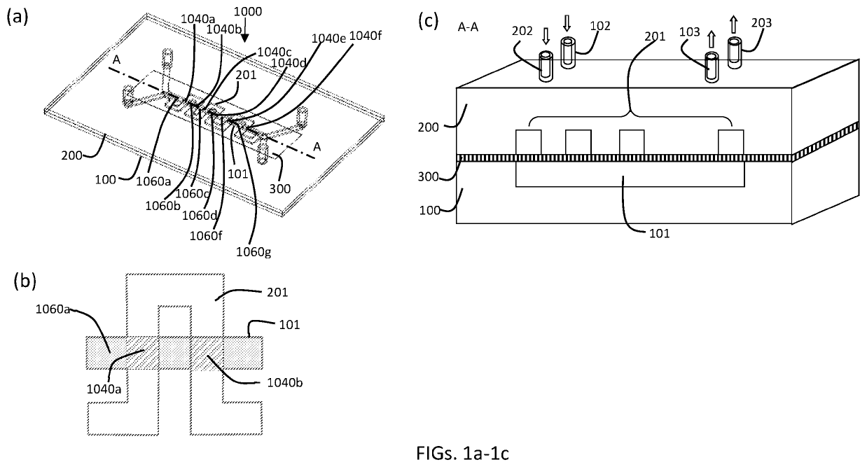

The microfluidic device includes multiple individual compartments separated by porous membranes (see, e.g., FIGS. 1a-1c, 2b, 3a-3c, 4a-4c, 5a-5c, 6a, 6b, 7a-7c, and 8a-8c). The disclosed microfluidic devices are preferably optical transparent, accommodating standard microscopy imaging and in-situ monitoring. Using color-tagged cells, activities and responses of cells in co-cultures can be tracked. Different from the models based on static cell culture using transwells, the models disclosed herein advances disease study (such as prostate disease study) using a complete organ involving complex tissue types and appropriate microenvironment. The microfluidic devices and models built therefrom offer high spatial resolution, high sensitivity, and controllability, and can be used for high-throughput screening in drug discovery.

For example, co-cultures of various types of tissue involved in a disease of interest (such as prostate diseases) can be implemented while providing physiological environment and communications among the different tissue types. For example, prostate disease related models, such as prostate tumor progression and invasion into heathy tissues within the organ, as well as into a different organ, can be investigated using various configurations of the disclosed microfluidic devices. Further, model based on the disclosed microfluidic devices promotes studies to dissect the interplay mechanisms in the disease progress, such as effects of microenvironments, stroma's role in tumor progression, and immune system responses. Accordingly, the model can be used to study and validate biological pathways in disease progression. Additionally, the model can be used for drug discovery. Desired local drug concentration and concentration distribution profile can be realized by balancing convection-diffusion among the compartments through fine-tuning culture medium flow rates and drug administering scheme.

### A. Device Configurations

Generally, the microfluidic device disclosed herein includes at least two layers and a porous membrane, each layer contains a channel. The first layer of the microfluidic device can have any suitable orientation relative to the second layer of the microfluidic device. In some forms, the first layer of the microfluidic device is in parallel with the second layer of the microfluidic device. The porous membrane is typically adjacent to each of and between the first and second layers of the microfluidic device. For example, as illustrated in FIGS. 1a and 1c, the microfluidic device 1000 includes two layers (a first layer 100 and a second layer 200) in parallel with each other, and a first porous membrane 300 adjacent to each of and between the first layer 100 and second layer 200.

The porous membrane sandwiched between the first and second layers of the microfluidic device separates the flows in each channel, while allows communication and interaction between cells in one or more of the channels of the microfluidic device. The porous membrane also allows diffusion of solutes from one or more channels into neighboring channels.

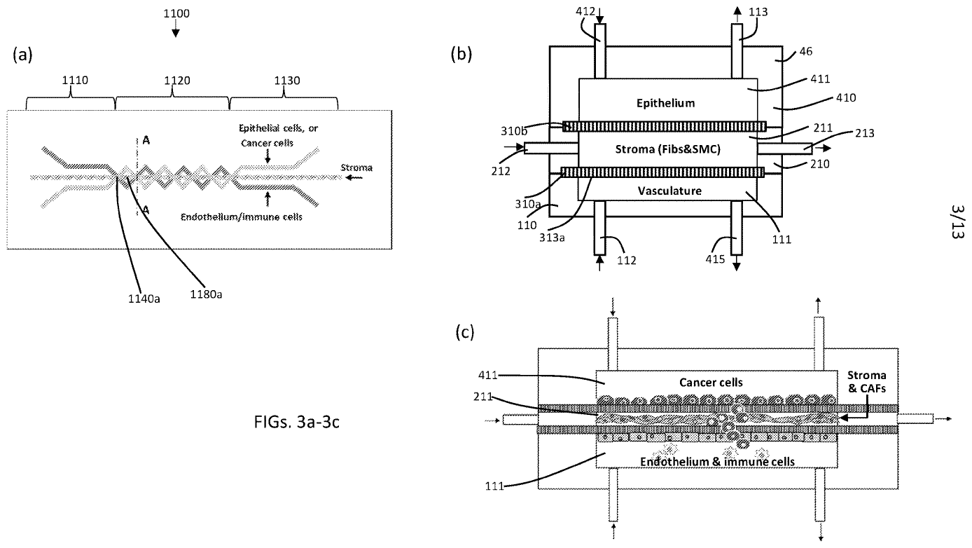

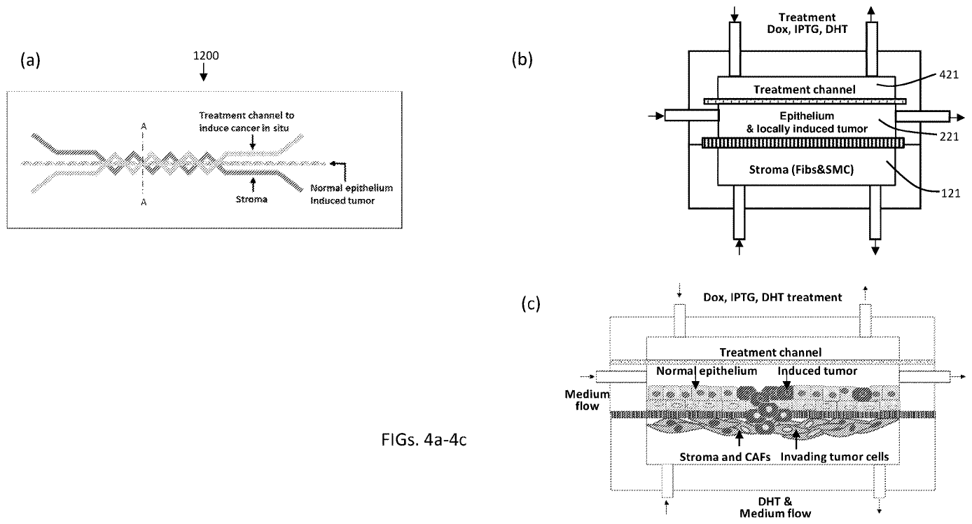

In some forms, the microfluidic device has more than two layers, each layer containing a channel. For example, the microfluidic device has three layers, four layers, five layers, six layers, seven layers, eight layers, nine layers, ten layers, etc. Typically, each pair of adjacent layers are separated by a porous membrane. For example, the microfluidic device includes three layers and two porous membranes (see, e.g., FIGS. 3a, 3b, 4a, and 4b). The first layer and second layers are in parallel with each other, and the first porous membrane is sandwiched between the first and second layers. The third layer is positioned on top of the second layer and in parallel with the first and second layers, and the second porous membrane is sandwiched between the third and second layers. The third channel of the third layer can have any suitable orientation relative to the channels of the first and second layers. For example, a region of the third channel of the third layer overlaps with a region of the second channel of the second layer. For example, as illustrated in FIGS. 3a and 3b, the microfluidic device 1300 includes three layers (a first layer 130, a second layer 230, and a third layer 430), and two porous layers (a first porous membrane 330a and a second porous membrane 330b). The first porous membrane 330a (such as having an average pore diameter of 0.4-10 microns) is adjacent to each of and between the first layer 130 and second layer 230, and the second porous membrane 330b (such as having an average pore diameter of 0.4-8 microns) is adjacent to each and between the second layer 230 and third layer 430.

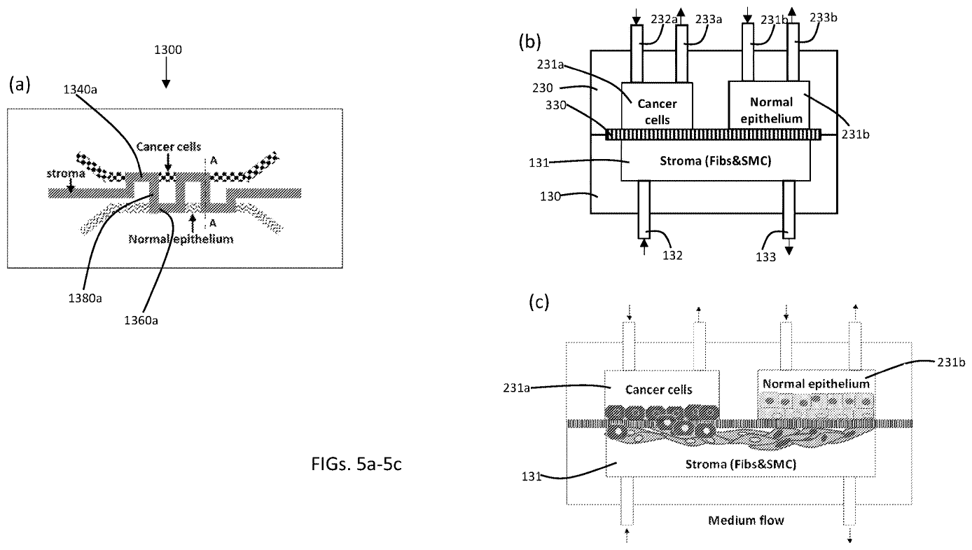

In some forms, one or more layers of the microfluidic device contains more than one channel. The channels in the same layer can have any suitable relative orientations. For example, two channels are contained in one layer and the two channels are in parallel with each other (see, e.g., FIGS. 5a and 5b). When two or more channels are contained in one layer, the channel in the layer adjacent to such layer, which is typically separated by a porous membrane, can have any suitable orientation relative to each of the channels of such layer. For example, one layer of the microfluidic device contains two channels (i.e., first and second channels), and the layer adjacent to this layer, separated by a porous membrane, contains one channel (i.e., third channel); the third channel may have one or more regions overlap with one of the first and second channels or both the first and second channels.

Preferably, each of the layers and porous membrane of the microfluidic device is formed of an inert, transparent material. Examples of transparent material suitable for forming the layers and porous membranes include, but are not limited to, polydimethylsiloxane (“PDMS”), polyester, acrylic, nylon, polycarbonate, polylactic acid, polyethylene terephthalate glycol, polyether ether ketone, polyether ketone ketone, polypropylene, and glass, and combinations thereof. The layers and porous membranes can be formed of the same material or at least two different materials. For example, the microfluidic device includes two or three layers and one or two porous membranes, where each of the layers is formed of PDMS or glass and each of the porous membrane is formed of polyester or glass. Glass can cut down on oxygen to create a reduced oxygen environment, based on the hypothesis that both the prostate gland, prostate tumors, and the bone have relatively low oxygenation relative to other organs.

Typically, at least one of the channels of the microfluidic device has a fluid inlet (see, e.g., FIG. 1c, 102) and a fluid outlet (see, e.g., FIG. 1c, 103). In some forms, each of one or more channels of the microfluidic device has a fluid inlet and a fluid outlet, and optionally one or more channels of the microfluidic device does not have a fluid inlet and/or a fluid outlet. In some forms, each channel of the microfluidic device includes a fluid inlet and a fluid outlet. The inlet and outlet are configured such that fluid flows in a certain direction through the channel. The flow direction of fluid in each channel can be the same or different. For example, the microfluidic device includes two channels, each including a fluid inlet and a fluid outlet. The inlet and outlet are configured such that fluid flows in a direction through the first channel and fluid flows in a second direction through the second channel. The first direction and the second direction can be the same or different. For example, the first direction is the same as the second direction.

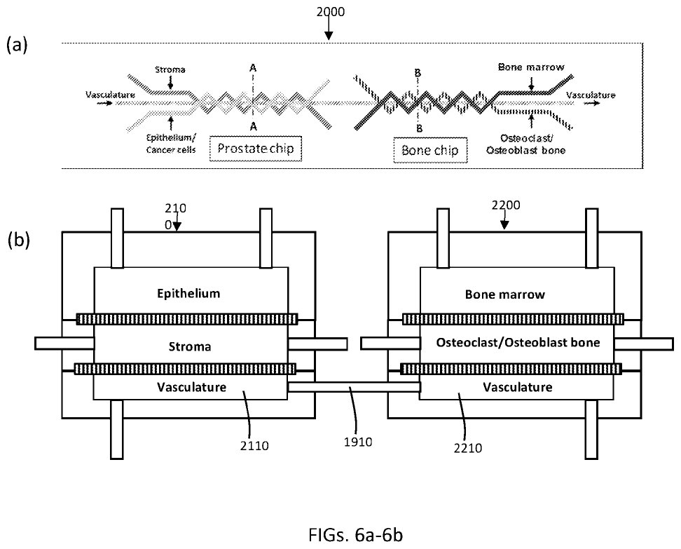

In some forms, two or more of the microfluidic devices can be fluidly connected with each other, such as via at least one channel of each of the connecting microfluidic devices, for studying interactions between two organs and/or cancer metathesis at a secondary organ. For example, as illustrated in FIG. 6b, a first microfluidic device 2100 is fluidly connected to a second microfluidic device 2200, where the first channel 2110 of the first microfluidic device is connected to the first channel 2210 of the second microfluidic device via a conduit 1910. Although not illustrated in the figures, it is understood that the microfluidic devices in a platform can be connected in any other desired way.

### 1. Porous Membrane

The porous membrane (when one porous membrane is included in the microfluidic device) or each porous membrane (when two or more porous membranes are included in the microfluidic device) has a top surface and a bottom surface. When sandwiched between two adjacent layers containing channel(s), the top and bottom surfaces of the porous membrane can function as the floor and the ceiling of the channels in the two adjacent layers of the microfluidic device, respectively. For example, as illustrated in FIG. 1c, the top surface 301 of the porous membrane 300 functions as the floor of the second channel 201 in the second layer 200, and the bottom surface 303 of the porous membrane 300 functions as the ceiling of the first channel 101 in the first layer 100. For example, as illustrated in FIG. 3b, a first porous membrane 310a is sandwiched between a first layer 110 and a second layer 210, while a second porous membrane 310b is sandwiched between the second layer 210 and a third layer 410. The top surface 311a of the porous membrane 310a functions as the floor of the second channel 211 in the second layer 210, and the bottom surface 313a of the porous membrane 310a functions as the ceiling of the first channel 111 in the first layer 110. The top surface 311b of the porous membrane 310b functions as the floor of the third channel 411 in the third layer 410, and the bottom surface 313b of the porous membrane 310 functions as the ceiling of the second channel 211 in the first layer 210. As such, when cells and/or tissues are placed inside the channel(s), they can be placed on the top and/or bottom surfaces of each pours membrane sandwiched between two adjacent layers to optionally facilitate transport of the cells and/or secreted solutes by the cells between neighboring channel(s) (see, e.g., FIGS. 3c and 5c).

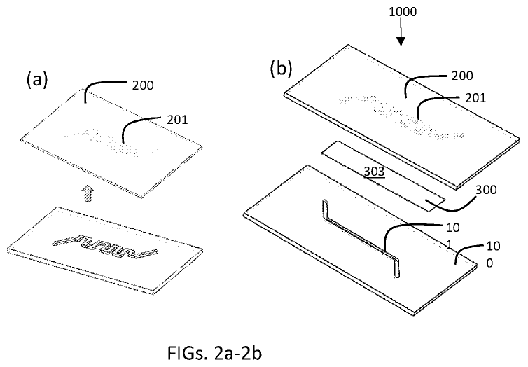

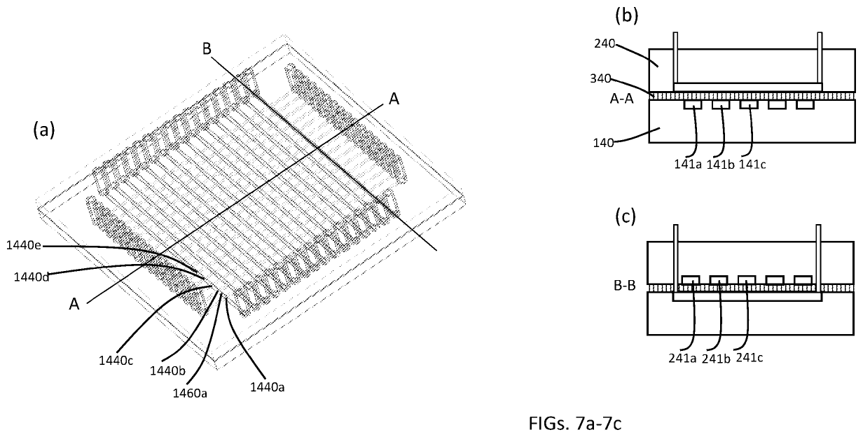

For example, each layer of the microfluidic device includes one channel (see, e.g., FIG. 2b, 101 and 201), two channels (see, e.g., FIG. 5b, 131, 231a, and 231b), or more than two channels, such as an array of channels (see, e.g., FIG. 7b, 141a, 141b, 141c, etc. and FIG. 7c, 241a, 241b, 241c, etc.). For example, as illustrated in FIG. 2b, the microfluidic device 1000 includes a first layer 100 containing a first channel 101 and a second layer 200 containing a second channel 201. For example, as illustrated in FIGS. 5a and 5b, the microfluidic device 1300 includes a first layer 130 containing a first channel 131, and the second layer 230 includes a second channel 231a and a third channel 231b. For example, as illustrated in FIGS. 7a-7c, the first layer 140 and second layer 240 each include an array of channels (141a, 141b, 141c, etc. in the first layer 140 and 241a, 241b, 241c, etc. in the second layer 240).

The porous membrane or each porous membrane (when two or more porous membranes are included) of the microfluidic device contains a plurality of pores. The pores in the plurality of pores of the porous membrane or each porous membrane can have the same diameter or two or more different diameters.

In some forms, the pores in the plurality of pores of the porous membrane or each porous membrane can have an average diameter ranging from about 0.4 micron to about 15 microns, from about 0.4 micron to about 10 microns, from about 0.4 micron to about 8 microns, from about 0.4 micron to about 5 microns, from about 0.4 micron to about 1 microns, from about 0.8 micron to about 15 microns, from about 1 micron to about 15 microns, from about 0.8 micron to about 10 microns, from about 1 micron to about 10 microns, from about 0.8 micron to about 8 microns, from about 0.8 micron to about 5 microns, from about 2 micron to about 15 microns, or from about 5 micron to about 15 microns, such as from about 0.4 micron to about 15 microns, from about 0.4 micron to about 10 microns, from about 0.4 micron to about 8 microns.

In some forms, the pores in the plurality of pores of the porous membrane have different diameters. The pores of different diameters can be in different groups/area, or with any desired arrangement on the porous membrane. For example, a first group of pores in the plurality of pores has a first average diameter ranging from about 0.4 micron to 10 micron, from about 0.4 micron to about 8 microns, from about 0.4 micron to about 5 microns, or from about 0.4 micron to about 1 microns, and a second group of pores in the plurality of pores has a second average diameter ranging from about 0.8 micron to about 15 microns, from 0.8 micron to about 10 microns, from about 1 micron to about 15 microns, or from about 1 micron to about 10 microns, and wherein the first diameter is different from the second diameter.

When two or more porous membranes are included in the microfluidic device, the pores in the plurality of pores of the first porous membrane can have the same or different average diameter compared to the pores in the plurality of pores of the second porous membrane. For example, the pores in the plurality of pores of the first porous membrane has the same average diameter of the pores in the plurality of pores of the second porous membrane, where the average diameter ranges from about 0.4 micron to about 15 microns, from about 0.4 micron to about 10 microns, from about 0.4 micron to about 5 microns, from about 0.4 micron to about 1 microns, from about 0.8 micron to about 15 microns, from about 1 micron to about 15 microns, from about 0.8 micron to about 10 microns, from about 1 micron to about 10 microns, from about 0.8 micron to about 8 microns, from about 0.8 micron to about 5 microns, from about 2 micron to about 15 microns, or from about 5 micron to about 15 microns. For example, the pores in the plurality of pores of the first porous membrane has a first average diameter ranging from about 0.4 micron to 15 micron, from about 0.4 micron to 10 micron, from about 0.4 micron to about 8 microns, from about 0.4 micron to about 5 microns, or from about 0.4 micron to about 1 microns, and the pores in the plurality of pores of the second porous membrane has a second average diameter ranging from about 0.8 micron to about 15 microns, from 0.8 micron to about 10 microns, from about 1 micron to about 15 microns, or from about 1 micron to about 10 microns, and wherein the first diameter is different from the second diameter.

### 2. Overlapping Regions and Non-Overlapping Regions

The microfluidic device includes a feed area (see, e.g., FIG. 3a, 1110), an exit area (see, e.g., FIG. 3a, 1120), and a material exchange area (see, e.g., FIG. 3a, 1130). Within the material exchange area, a region of the channel of the first layer overlaps with a region of the channel of the second layer. For example, the channel of the first layer and the channel of the second layer can have one overlapping region or two or more discrete overlapping regions. The overlapping region or each of the two or more overlapping regions can have any suitable shape and dimensions. The geometry of the overlapping regions in each channel can be the same or different, where each overlapping region can be in planar (squared, circular, etc.), or in 3D (dome, cone, truncated cone, cylinder, etc.). Typically, within the material exchange area, the channel of the first layer also has one region or two or more regions that do not overlap with any region of the channel of the second layer.

In some forms, the channel or each channel (when two or more channels are present) in a layer is aligned with the channel or each channel (when two or more channels are present) in its neighboring layer or each neighboring layers, such that a region of the channel or each channel in the layer overlaps with a region of the channel or each channel in its neighboring layer or each neighboring layer, and a region of the channel or each channel in the layer does not overlap with any region of the channel or each channel in its neighboring layer or each neighboring layer.

For example, within the material exchange area of a microfluidic device, a first channel in a first layer is aligned with a second channel in a second, adjacent layer, such that a first region (see, e.g., FIG. 1b, 1040a) of the first channel in the first layer overlaps with a second region (see, e.g., 1050a, not visible in FIG. 1b) of the second channel in the second layer, and a third region (see, e.g., FIG. 1b, 1060a) of the first channel does not overlap with any region of the second channel in the material exchange area.

For example, within the material exchange area of a microfluidic device, a first channel in a first layer is aligned with a second channel and a third channel in a second, adjacent layer, such that a first region (see, e.g., FIG. 5a, 1340a) of the first channel in the first layer overlaps with a second region (see, e.g., 1350a not visible in FIG. 5a) of the second channel in the second layer, a third region (see, e.g., FIG. 5a, 1360a) of the first channel overlaps with a fourth region (see, e.g., 1370a not visible in FIG. 5a) of the third channel in the second layer, and a fifth region (see, e.g., FIG. 5a, 1380a) of the first channel does not overlap with any region of the second and third channel in the material exchange area.

For example, within the material exchange area of a microfluidic device, a second channel in a second layer (which is between a first and third layer) is aligned with a first channel in the first layer and with a third channel in the third layer, such that a first region (see, e.g., FIG. 3a, 1140a) of the second channel overlaps with a second region (see, e.g., 1150a not visible in FIG. 3a) of the first channel, the first region (see, e.g., FIG. 3a, 1140a) of the second channel also overlaps with a fourth region (see, e.g., 1170a not visible in FIG. 3a) of the third channel, and a fifth region (see, e.g., FIG. 3a, 1180a) of the second channel does not overlap with any region of the first and third channels in the material exchange area. Within the material exchange area of a microfluidic device, when a channel in a layer overlaps with channels in two adjacent layers (e.g., such layer is sandwiched between the two adjacent layers, separated by two porous membranes, see, e.g., FIG. 3a and 3b), the region of such channel that overlaps with the channels in the two adjacent layers can be the same or different. For example, as illustrated in FIG. 3b, the regions of the second channel that overlap with the first and third channels are the same. Although not illustrated in FIGS. 3a and 3b, in some forms, the regions of the second channel that overlap with the first and third channels may be different.

For example, within the material exchange area of a microfluidic device, each channel (see, e.g., FIG. 7b, 141a) in a first array of channels in a first layer is aligned with each channel in a second array of channels in a second layer, such that the channel in the first array has a region (see, e.g., FIG. 7a, 1440a, 1440b, 1440c, 1440d, 1440e, etc.) that overlaps with a region (see, e.g., 1450a, 1450b, 1450c, 1450d, 1450e, etc. in FIG. 7a, which overlaps with 1440a, 1440b, 1440c, 1440d, 1440e, etc. and thus not labeled) of each channel in the second array of channels, and a region (see, e.g., FIG. 7a, 1460a) that does not overlap with any region of any channel in the second array.

Within the material exchange area of the microfluidic device, the channel or each channel in a layer may include more than one region that overlaps with the channel or each channel in its neighboring layer or each neighboring layer, such as two or more overlapping regions, for example, two, three, four, or five, or more than five overlapping regions (see, e.g., FIG. 1a, 1040a, 1040b, 1040c, 1040d, 1040e, 1040f, and 1040g); and/or more than one region that does not overlap with any region in its neighboring layer or each neighboring layer, such as two or more non-overlapping regions, for example, two, three, four, or five, or more than five non-overlapping regions (see, e.g., FIG. 1a, 1060a, 1060b, 1060c, 1060d, 1060e, 1060f, and 1060g). In some forms, the microfluidic device can include one or more reservoirs in fluid connection with one or more channels in at least one layer of the microfluidic device, optionally positioned at the overlapping region(s) of the channel(s).

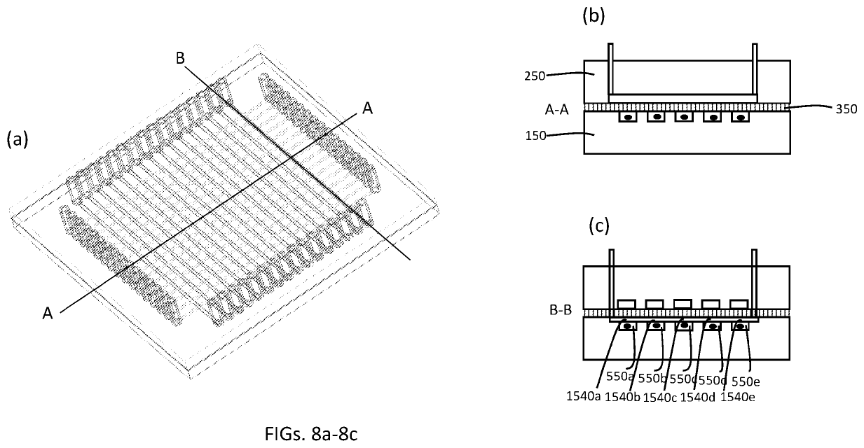

In some forms, one or more layers of the microfluidics device contains a multiple-channel array (see, e.g., FIGS. 7a and 8a). The array of channels in each layer can have any suitable relative orientations. For example, the channels are in parallel with each other in the array. When an array of channels is contained in one layer, the channels in the layer adjacent to such layer, which is typically separated by a porous membrane, can have any suitable orientation relative to the channel array of such layer. For example, one layer of the microfluidic device contains a 16-channel array, and the layer adjacent to this layer, separated by a porous membrane, contains a 16-channel array; the two channel arrays may have 16×16 overlapping regions. When one or more layers of the microfluidic device contains channel arrays, the layer containing multiple channels can be arranged in any suitable position or orientation in the microfluidic device. The number of the channels in the channel array in the first layer can be the same as or different from the number of channels in the channel array in the second layer. The geometry and dimensions of the channels in the channel array in the first layer can be the same as or different from the channels in the channel array in the second layer.

In some forms, the channels in a channel array in a first, lower layer (see, e.g., FIGS. 8b and 8c, 150) of a microfluidic device are fluidly connected to reservoirs at the overlapping regions of the channels (see, e.g., FIG. 8c, 1540a, 1540b, 1540c, 1540d, and 1540e). The reservoirs are in fluid communication with the channel and can be positioned in any suitable position relative to the channel, such as fluidly connected to the floor of the channel optionally at each overlapping region of the channel (see, e.g., FIG. 8c, 1540a, 1540b, 1540c, 1540d, and 1540e). These reservoirs can be configured to retain 3D tissues, such as tumor spheroids or organoids, in the co-culture of the disease model, and/or to retain drug capsules, releasing drug at an extended period of time with local concentration gradient. The reservoirs can have any suitable geometry and dimensions, and can be selected based on the specific function. For example, the reservoirs has a volume larger than the volume of the carrier(s) in the drug carrier medium and a height that is larger than the height of the channel to which it is connected, to facilitate deployment of the carriers when the drug carrier medium flown over/through.

### 3. Dimensions

a. Layer/Porous Membrane Height

Each of the layers and porous membranes of the microfluidic device can have any suitable height, depending on the specific applications of the microfluidic device. For example, each of the layers and porous membranes of the microfluidic device has a height ranging from about 50 microns to about 4 mm, from about 100 microns to about 4 mm, from about 50 microns to about 2 mm, from about 100 microns to about 2 mm, from about 50 microns to about 1 mm, from about 100 microns to about 1 mm, from about 10 microns to 1 mm, from about 10 microns to about 4 mm, from about 10 microns to about 2 mm, from about 50 microns to about 500 microns, from about 100 microns to about 200 microns, from about 10 microns to 100 microns, or from about 15 microns to about 50 microns. The layers and porous membranes can have the same height or at least two different heights. For example, the microfluidic device includes two or three layers and one or two porous membranes, where each of the layers has a height ranging from about 50 microns to about 500 microns, and each of the porous membranes has a height ranging from about 15 microns to about 50 microns. In these forms, the layers can have the same height or at least two different heights and the porous membranes can have the same height or at least two different heights. In some forms, a layer of larger height, such as a height of 2 mm or 4 mm, is particular useful for applications using hydrogels with organoids, tumor spheroids, embedded cells, etc.

### B. Channel Dimensions and Shapes

The channels of the microfluidic device can have any suitable dimensions and shapes, and can be arranged in any suitable relative positions, as long as desired overlapping region(s) and non-overlapping region(s) of two channels in neighboring layers can be achieved in the material exchange area. Suitable shapes for each channel in the microfluidic device include, but are not limited to, straight and meander, such as a bent, curved, or serpentine shape. For example, each channel in the microfluidic device is independently a straight or meandering channel, such as a channel having a bent, curved, or serpentine configuration.

For example, when the microfluidic device includes two channels, within the material exchange area, one channel is substantially straight (along the length) and the other channel can have a wavy shape, such that one or more regions of the straight channel overlap with one or more regions of the wavy channel; and one or more regions of the straight channel do not overlap with any region of the wavy channel, in the material exchange area.

For example, when the microfluidic device includes three channels, within the material exchange area, a first channel in the first layer can be wavy (see, e.g., FIG. 5a, 131), and a second and third channels in the second layer can each be substantially straight (along the length) (see, e.g., FIG. 5a, 231a and 231b), such that one or more regions of the first channel overlap with one or more regions of each of the second and third channels; and one or more regions of the first channel do not overlap with any region of the second and third channels, in the material exchange area. For example, when the microfluidic device includes three channels, within the material exchange area, the second channel (see, e.g., FIG. 3a, 211) in the second layer can each be substantially straight (along the length) and the first (see, e.g., FIG. 3a, 111) and third (see, e.g., FIG. 3a, 411) channels in the first and third layers, respectively, can be wavy, such that two or more regions of the second channel overlap with two or more regions of each of the second and third channels, and two or more regions of the second channel do not overlap with any region of each of the first and third channels, in the material exchange area.

Examples of wavy shapes that can be used to form the wavy channel include, but are not limited to, serpentine, sinewave, and triangle-wave, and combinations thereof. The width of the channel can be uniform or varied.

In some forms, each channel in the microfluidic device can independently has a width ranging from about 50 microns to about 4 mm, from about 100 microns to about 4 mm, from about 50 microns to about 2 mm, from about 100 microns to about 2 mm, from about 50 microns to about 1 mm, from about 100 microns to about 1 mm, from about 10 microns to 1 mm, from about 10 microns to about 4 mm, from about 10 microns to about 2 mm, from about 50 microns to about 500 microns, from about 100 microns to about 200 microns, from about 10 microns to 100 microns, or from about 15 microns to about 50 microns; and/or a length ranging from 5 mm to 100 mm, from 5 mm to 90 mm, from 5 mm to 80 mm, from 5 mm to 70 mm, from 5 mm to 60 mm, from 5 mm to 50 mm, from 5 mm to 40 mm, from 5 mm to 30 mm, from 5 mm to 20 mm, from 5 mm to 10 mm, from 10 mm to 100 mm, from 10 mm to 90 mm, from 10 mm to 80 mm, from 10 mm to 70 mm, from 10 mm to 60 mm, from 10 mm to 50 mm, from 10 mm to 40 mm, from 10 mm to 30 mm, or from 10 mm to 20 mm.

The height and/or width of each channel can be uniform along the length of the channel or varied along the length of the channel. In some forms, the height of the channel is uniform along the length of the channel. In some forms, the height of the channel can vary. For example, the height of the channel at an overlapping region (or regions) is larger than the rest of the channel. Additionally or alternatively, the width of the channel can the uniform or be varied along the length of the channel. For example, the width of one or more channels in the microfluidic device is larger at the overlapping region(s) than the rest of the channel.

### B. Organ-on-a-Chip Platforms

Organ-on-a-chip platforms based on the microfluidic devices are also described. The platforms can be formed using any of the microfluidic devices described herein. For example, the platform may be formed using any one, or two or more microfluidic devices described herein. When two or more microfluidic devices are included in the platform, at least one channel of each microfluidic device can be in fluid connection with another microfluidic device in the platform.

The platform contains at least one type of tissue in at least one channel of the microfluidic device. In some embodiments, the organ-on-a-chip platform contains two or more types of tissue in a single channel. Optionally, one channel contains fibroblasts and smooth muscle cells in suitable ratio to mimic what is seen in vivo, such as a ratio of approximately 80% fibroblasts to 20% smooth muscle cells. In some embodiments, the platform includes a microfluidic device described herein and at least two different types of tissues, where the two different types of tissues can be in the same channel or different channels. The amount of tissue in each channel can be assessed based on the number of the cells that are initially placed within the channel, and by measuring markers known to be associated with each cell type at the end.

For example, two different types of tissues are in the channels of the microfluidic device, wherein at least 70%, at least 80%, or at least 90% of the first type of tissue is in a first channel, and at least 70%, at least 80%, or at least 90% of the second type of tissue is in a second channel. For example, two different types of tissues are in the channels of the microfluidic device, wherein at least 90% of the first type of tissue and at least 90% of the second type of tissue are in the same channel.

For example, three different types of tissues are in the channels of the microfluidic device, wherein at least 70% of the first type of tissue is in a first channel, at least 20% of the second type of tissue is in the first channel, and at least 90% of the third type of tissue is in a second channel. For example, three different types of tissues are in the channels of the microfluidic device, wherein at least 90% of the first type of tissue is in a first channel, at least 90% of the second type of tissue is in a second channel, and at least 90% of the third type of tissue is in a third channel.

For example, four different types of tissues are in the channels of the microfluidic device, wherein at least 90% of the first type of tissue is in a first channel, at least 90% of the second type of tissue is in a second channel, at least 90% of the third type of tissue is in the second channel, and at least 90% of the fourth type of tissue is in a third channel.

### C. Exemplary Devices and Platforms

Exemplary microfluidic devices and organ-on-a-chip platforms formed therefrom are illustrated in FIGS. 1a-1c, 2b, 3a-3c, 4a-4c, 5a-5c, 6a, 6b, 7a-7c, and 8a-8c.

It is understood that each of these exemplary microfluidic devices can be modified to incorporate other configurations (e.g., overall device configuration, such as arrangement of layers, shape, size, etc.; channel configuration, such as channel arrangement, shape, size, etc.; inlet and outlet arrangement, etc.), other dimensions, other orientations (e.g., channel orientation, inlet and outlet orientation, etc.), and other tissue types, depending on the specific application.

For example, any one of the channels in the devices exemplified in FIGS. 1a-1c, 2b, 3a-3c, 4a-4c, 5a-5c, 6a, 6b, 7a-7c, and 8a-8c can be modified to be in a straight/wavy shape or in a different wavy shape.

For example, any one of the channels in the devices exemplified in FIGS. 1a-1c, 2b, 3a-3c, 4a-4c, 5a-5c, 6a, 6b, 7a-7c, and 8a-8c can be modified to include at least two wavy channels and one straight channel. This can facilitate visualization of migration in the different channels. In such a setup, the first channel may be loaded with epithelial/cancer cells, the second channel (which is in the middle layer) can be loaded with fibroblasts/smooth muscle cells, and the third channel can be loaded with endothelial cells (see, e.g., FIG. 3c). The third channel can have a shorter height compared to the first and second channels to create increased sheer stress/flow velocity to mimic vasculature physiology. Such an arrangement mimics prostate organ/tumor architecture.

For example, any one of the devices exemplified in FIGS. 1a-1c, 2b, 3a-3c, 4a-4c, 5a-5c, 6a, 6b, 7a-7c, and 8a-8c can be modified to include three or more channels in three layers, having any other shapes and/or in any other arrangements.

For example, the organ-on-chip platforms exemplified in FIGS. 6a and 6b can be modified to use any other and/or additional microfluidic devices described herein for study of interaction between any other organs or disease models.

### II. Methods of Making

The microfluidic devices and organ-on-a-chip platforms disclosed herein can be produced using methods known in the art. For example, the microfluidic devices can be prepared using photolithography, a specific example of which is described in the Example section below. The organ-on-a-chip platforms can be prepared by placing cells in the respective channels of the microfluidic devices and flowing culture mediums through the channels containing the cells. The specific types of cells and their placement in the channels of the microfluidic devices depend on the specific disease or organ being studied. An exemplary procedure for preparing prostate cancer models based on the disclosed microfluidic device is described in the Example section below.

### III. Methods of Use

The disclosed microfluidic devices have a wide variety of uses in biomedical applications, such as for constructing a disease model (e.g., bio-Engineered human prostate disease model), studying disease mechanisms, and studying how disease influences other organs, etc.

Prostate disease related models, such as prostate tumor progression and invasion into heathy tissues within the organ, as well as into different organs, can be investigated using various configurations of the devices. Role of infectious agents in prostatitis or mechanisms of benign prostate hyperplasia development can be interrogated. Additionally, the disclosed microfluidic devices can be used to dissect the interplay mechanisms in various prostate diseases, including the effects of microenvironmental factors such as stroma, vascular, sensory neurons, and immune system responses. Physical components, such as variations in matrix, pH, and oxygen tension can be modeled in prostate disease states. The model can be used to study and validate genetic and biological pathways in disease progression.

Further, the disclosed microfluidic devices can be used for drug discovery, drug screening, and drug dose regimen test, Desired local drug concentration and concentration distribution profiles can be realized by balancing convection-diffusion among the compartments through fine-tuning cell culture medium flow rates and drug administering schemes. The devices described herein can be used for high throughput screening in drug discovery.

### A. Constructing Disease Model

In some forms, the microfluidic device disclosed herein can be used for constructing a disease model. The method generally includes (i) placing cells in one or more channels of the microfluidic device and (ii) flowing suitable culture medium through each channel containing the cells to form tissue. The cells may be placed in each of the channels of the microfluidic device, or selectively placed in one or more channels of the microfluidic device but not all. The cells being placed in one channel can be a single type or a mixture of different types, in 2D form or 3D form. For example, the cells can be placed in the channel(s) of the microfluidic device in a 2D form (for example, in the form of a monolayer) or a 3D form (for example, in the form of organoids, spheroids, one or more types of cells embedded in hydrogel or ex vivo tissue, etc.).

Each channel in the microfluidic device may contain a different type of cells or the same type of cells, or a combination thereof. For example, each channel in the same layer may contain the same or different type of cells; or at least one channel contains a type of cells that is different from the other channels in the same layer. In some forms, at least one channel in one or more layers of the microfluidic device does not contain any cells. For example, in step (i) in the channel(s) of each layer of a microfluidic device, a different type of cells is placed. Alternatively, in step (i) in the channel(s) of at least one layer of the microfluidic device, a first type of cells is placed, and in the channel(s) of one or more layers, no cells are placed. The placement of cells in the microfluidic device can be in any suitable pattern, and can be selected based on the specific cells, tissues, disease models, dimensions of the channels, configuration of the channels, etc.

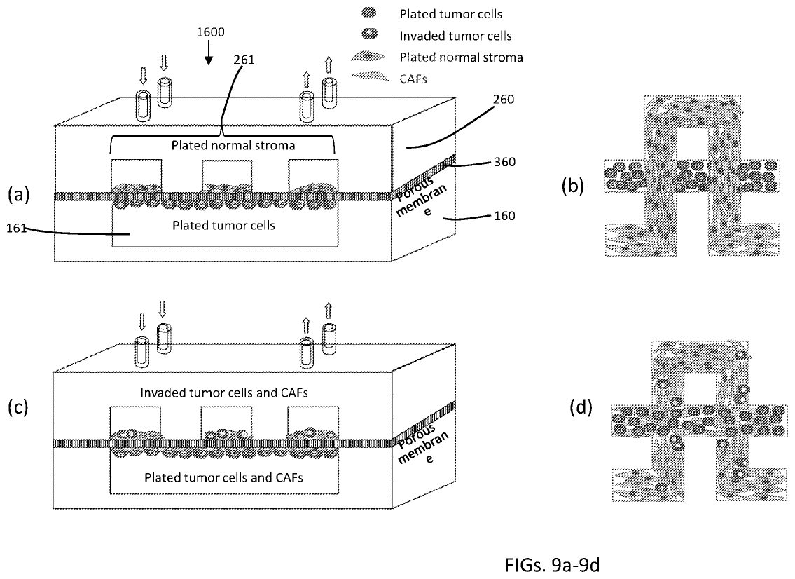

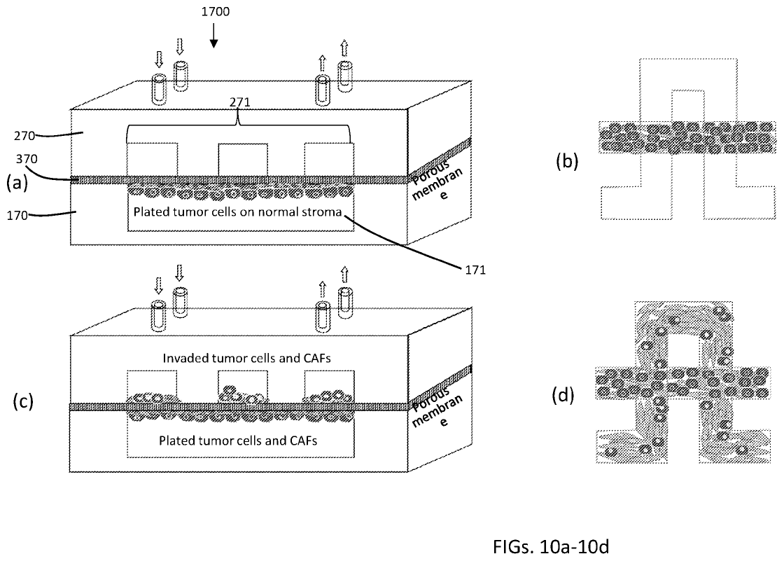

For example, the microfluidic device contains two layers, and each layer contains one or more channels. In these forms, in step (i) in each channel of each layer, a different type of cells is placed (see, e.g., FIG. 5b, a first type of cells is placed in the first channel 131 of the first layer 130 of the microfluidic device 1300, a second type of cells is placed in the second channel 231a of the second layer, and a third type of cells is placed in the third channel 231b of the second layer; and see also, e.g., FIG. 9a, a first type of cells is placed in the first channel 161 of the first layer 160 of the microfluidic device 1600, and a second type of cells is placed in the second channel 261 of the second layer 260). In some forms, more than one type of cells can be placed in a single channel. In some forms, in step (i) in the channel(s) of the first layer, one or more types of cells are placed (see, e.g., FIG. 10a, tumor cells placed on a layer of stroma in the first channel 171), and in the channel(s) of the second layer, no cells are placed (see, e.g., FIG. 10a, no cells in the second channel 271).

For example, the microfluidic device contains three layers, and each layer contains one or more channels. In these forms, in step (i) in each channel of each layer, a different type of cells is placed (see, e.g., FIG. 3b, a first type of cells is placed in the first channel 111 of the first layer 110 of microfluidic device 1100, a second type of cells is placed in the second channel 211 of the second layer 210, and a third type of cells is placed in the third channel 411 of the third layer 410). Alternatively, in step (i) in the channel(s) of at least one layer, one or more types of cells are placed, and the channel(s) of at least one layer does not contain any cells. For example, in step (i) in the channel(s) (see, e.g., FIG. 4b, channel 121) of the first layer, a first type of cells is placed; in the channel(s) (see, e.g., FIG. 4b, channel 221) of the second layer, a second type of cells is placed; and no cells are placed in the channel(s) (see, e.g., FIG. 4b, channel 421) of the third layer. In some forms, more than one type of cells can be placed in a single channel.

Typically, following step (ii), at least one type of tissue, preferably two different types of tissues, is/are formed in the channels of the microfluidic device. For example, when a different type of cells is placed in each channel, a different type of tissue is formed in each channel following step (ii). When two or more different types of cells are placed in a single channel, two or more different types of tissues are formed in the single channel following step (ii). The relative amounts of the type of tissue formed in each channel can be determined at desired time points following step (ii), such as 1 day, 2 days, 3, days, 4 days or 5 days following step (ii), to determine the initially formed tissue composition. When only one type of cell is placed in a given channel, the initial composition of the tissue formed in the channel is typically predominantly one type of tissue, i.e. initially at least 90% of at least one type of tissue is in the channel.

Optionally, one or more cells placed in one channel infiltrate into the adjacent channel (which does not have the same cells placed therein in step (i)) via the pores of the porous membrane. Optionally, the method further includes, prior to step (i), coating the channels with a suitable matrix. The matrix used for coating each channel is known and can be selected based on the specific cell types cultured in the channel. Specific examples of the matrix suitable for coating the channel prior to cell placement are described in the Examples section below.

For example, the microfluidic device used for constructing a disease model includes at least two layers, each layer containing a channel, and a porous membrane sandwiched between the two layers. In these forms, the method includes: (i) placing one or more types of cells in the first channel and optionally one or more types of cells in the second channel; and (ii) flowing a first culture medium through the first channel and optionally a second culture medium through the second channel. For example, one channel of the microfluidic device initially contains epithelial cells and the other channel initially contains fibroblasts or smooth muscle cells, or a combination of both therein. For example, one channel of the microfluidic device initially contains normal fibroblasts and the other channel initially contains tumor cells (see, e.g., FIG. 9a). For example, one channel of the microfluidic device initially has tumor cells placed on a monolayer of fibroblasts therein and another channel does not have any cells placed therein (see, e.g., FIG. 10a).

The cancer cells and fibroblasts/smooth muscle cells can be placed in channels of any suitable position and shape.

In step (ii), cells are cultured to form tissues under any suitable conditions. For example, step (ii) is performed at about 37° C. for a time period ranging from 3 to 21 days or from 7 to 18 days. Typically, in step (ii), each of the first and second culture media has an adequate input flow rate such that normalized concentration 10% (vol) or less of the medium, including secreted factors by cells (such as PSA, TGFβ, PDGF, TNFα, exosomes, Wnts, cytokines, FGF, KGF, HGF, etc.) in the first channel diffuses through the first porous membrane into the second channel, and optionally 10% (vol) or less of the medium, including secreted factors by cells, in the second channel, diffuses through the porous membrane into the first channel. In addition to flow rate, other conditions, such as shear stress, diffusion level, and coating/matrix, etc. are also selected to establish the appropriate physiological microenvironment for proper cell growth, cross-talks among the tissues, and other cell activities.

Following step (ii), at least one type of tissue, preferably two different types of tissues, is/are formed in the channels of the microfluidic device. Optionally, one or more cells of the tissue or each tissue are in the adjacent channel (which does not have the same cells placed therein in step (i)) (see, e.g., FIGS. 9b and 10b).

For example, following step (ii), two different types of tissues are formed in the channels of the microfluidic device, wherein at least 90% of the first type of tissue is in the first channel, and at least 90% of the second type of tissue is in the second channel. Optionally, one or more cells of the first type of tissue is in the second channel and/or one or more cells of the second type of tissue is in the first channel. The amount of tissue in each channel can be assessed using the number of cells that are initially placed within the channel, and by measuring markers known to be associated with each cell type at the end.

For example, following step (ii), two different types of tissues are formed in the channels of the microfluidic device, wherein at least 90% of the first type of tissue and at least 90% of the second type of tissue are in the first channel, and the second channel does not have more than 10% of the first or second type of tissue. Optionally, one or more cells of the first type of tissue and/or one or more cells of the second type of tissue is in the second channel. For example, when fibroblasts and smooth cells are co-cultured in one channel, the two cells can have an 80/20% ratio to mimic what is seen in vivo.

For example, following step (ii), three different types of tissues are formed in the channels of the microfluidic device, wherein at least 90% of the first type of tissue is in the first channel, at least 90% of the second type of tissue is in the first channel, and at least 90% of the third type of tissue is in the second channel. Optionally, one or more cells of the first type of tissue and/or one or more cells of the second type of tissue is in the second channel, and/or one or more cells of the third type of tissue is in the first channel.

Optionally, the method further includes, prior to step (i), coating the channels with a suitable matrix. first channel with a first matrix and coating the second channel with a second matrix.

In some forms, one layer of the microfluidic device may include two or more channels having any suitable shape and orientation relative to the other channels, or the microfluidic device includes a third layer and a second porous membrane each having any suitable shape and orientation relative to the other layers and channels, such as those described above and exemplified in the Figures. In these forms, it is understood that any cells of a suitable type (such as cells of a single type or cells of two or more different types, for example, endothelial cells, immune cells, pericytes, etc.) can be placed into the additional channel(s) and a suitable culture medium can flow therethrough in step (ii) to form a tissue and optionally one or more cells placed therein can diffuse into the adjacent channel in step (ii). Similarly, in step (ii), the culture media has an adequate flow rate such that normalized concentration of 10% (vol) or less of the medium diffuses through the porous membrane into the adjacent channel. Further, other conditions, such as shear stress, diffusion level, and coating/matrix, etc. are also selected to establish the appropriate physiological microenvironment for proper cell growth, cross-talks among the tissues, and other cell activities. In these forms, following step (ii), two or more different tissues are formed in the channels of the microfluidic device, such as three different tissue types, four different tissue types, five different tissue types, etc. Alternatively, one or more of such additional channels can be left empty (i.e., not having cells placed therein in step (i)), and one or more cells placed in an adjacent channel can infiltrate into the empty channel in step (ii).

### B. Drug Regime Screening

In some forms, the microfluidic device disclosed herein can be used for drug screening, such as to determine dosing regimen. Any drugs can be tested using the microfluidic device, such as anti-cancer agents, anti-inflammatories, growth factors, steroids, antibodies, etc.

Generally, the method includes step (i) placing cells in one or more channels of the microfluidic device and step (ii) flowing culture medium through one or more channels, as described above to first construct a disease model, and further include (iii) flowing a drug solution or suspension, or flowing a medium containing one or more carriers containing one or more drugs (also referred to herein as “drug carrier medium”), through one or more channels. The drug carrier medium contains a suitable medium and one or more drug carriers that contain a suitable concentration of drug(s), such as a high concentration of drugs, where the drugs can be enclosed/embedded in the carriers, such as small-volume capsules and/or beads. The drug carrier can be formed by any suitable materials, such as gelatin, collagen, Matrigel, agarose, alginate, etc.

When a drug carrier medium is used in the method, the method may further include after step (iii), flowing a cell culture medium (without any drugs) through the first channel and/or the second channel.

In step (iii), the drug solution or suspension can be administered in channels that initially contain at least 70%, at least 80%, or at least 90% of one type of tissues (or of a combination of two or more tissues, such as a combination of smooth muscle cells and fibroblasts), or in channels that do not have cells placed therein in step (i) but may have one or more cells infiltrated therein from adjacent channels following step (ii). Typically, the drug solution or suspension has a sufficient flow rate and initial concentration such that a concentration gradient is produced along the channel it is administered into due to the intake of the drugs by cells in the device.

The method may further include during or after step (iii), measuring cell responses to the drug. Examples of cell responses that can be measured include, but are not limited to, cell death as indicated by release of solutes (such as LDH), or measured using live/dead assay or cell counting; molecular events (such as TUNEL, LC3/autophagy) by staining; loss of specific pathway signaling targeted by drug either through the use of cells reporters within the cells or by immunostaining; loss of cell adhesion (such as loss of adhesion to ECM as measured by ratio of area of attached cell layer to area of cell original surface before drug treatment (fully confluent layer); loss of cell adhesion to neighboring cells (such as measured by numbers of individual intact cell groups, where before drug treatment, the cells formed a full layer); change in cell migration rate or direction-measured by time-dependent imaging of cells; increased/decreased expressions in certain proteins as measured by ELISA, PCR, flow cytometry, immunostaining, etc.

The flow of the drug solution or suspension in each channel can have any suitable direction and can be the same or different. Further, drug concentration, treatment period, dosage, input channel(s) can be controlled by designing medium control flow scheme.

When a drug carrier medium is used in the method, the microfluidic device can include one or more reservoirs connected to one or more channels in at least one layer of the microfluidic device. For example, the microfluidic device includes an array of reservoirs connected to each channel in the first layer, located at each overlapping region of each channel (see, e.g., FIG. 8c, 1540a, 1540b, 1540c, 1540d, and 1540e) in the first layer. The reservoirs are in fluid communication with the channel and can be positioned in any suitable position relative to the channel, such as fluidly connected to the floor of the channel, such as at the overlapping regions of the channel (see, e.g., FIG. 8c, 1540a, 1540b, 1540c, 1540d, and 1540e). The reservoirs can have any suitable geometry and dimensions, as long as it has a volume larger than the volume of the carrier(s) in the drug carrier medium and a height that is larger than the height of the channel to which it is connected, to facilitate deployment of the carriers when the drug carrier medium flown over/through. In these forms, the carriers in the drug carrier medium can be deployed by flowing the drug carrier medium through the channel connecting to the reservoirs at overlapping regions. The carriers containing the drugs can be then retained in the reservoirs (see, e.g., FIG. 8c, 550a, 550b, 550c, 550d, and 550e). The drug carriers retained in the reservoirs can release drug locally at the overlapping regions in flowing culture medium that does not contain any drugs.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

The disclosed devices and methods can be further understood through the following enumerated paragraphs.
1. A microfluidic device comprising:
   at least two layers, each layer comprising a channel; and
   a first porous membrane,
   wherein the first layer is in parallel with the second layer,
   wherein the first porous membrane is adjacent to each of and between the first and second layers,
   wherein the microfluidic device comprises a material exchange area,
   wherein within the material exchange area, a first region of a first channel overlaps with a second region of a second channel, and
   wherein a third region of the first channel does not overlap with any region of the second channel in the material exchange area.
2. The microfluidic device of paragraph 1, wherein the microfluidic device comprises a feed area and an exit area, wherein within the feed area, each channel comprises a fluid inlet, and within the exit area, each channel comprises a fluid outlet, wherein each of the fluid inlets and fluid outlets are configured such that fluid flows in a first direction through the first channel and fluid flows in a second direction through the second channel, and wherein the first direction and the second direction are the same or different, optionally wherein the first direction is the same as the second direction.
3. The microfluidic device of paragraph 1 or 2, wherein the first porous membrane comprises a plurality of pores, and optionally wherein the pores in the plurality of pores have the same average diameter or comprise two or more groups, where each group of pores has a different average diameter.
4. The microfluidic device of paragraph 3, wherein the plurality of pores have an average diameter ranging from about 0.4 micron to about 15 microns, from about 0.4 micron to about 10 microns, from about 0.4 micron to about 5 microns, from about 0.4 micron to about 1 microns, from about 0.8 micron to about 15 microns, from about 1 micron to about 15 microns, from about 0.8 micron to about 10 microns, from about 1 micron to about 10 microns, from about 0.8 micron to about 8 microns, from about 0.8 micron to about 5 microns, from about 2 micron to about 15 microns, or from about 5 micron to about 15 microns; or
   wherein the plurality of pores comprises two or more groups, wherein a first group of pores has a first average diameter ranging from about 0.4 micron to 5 micron, and a second group of pores has a second average diameter ranging from 0.8 micron to about 15 microns, and wherein the first average diameter is different from the second average diameter.
5. The microfluidic device of any one of paragraphs 1-4, wherein each of the first layer, the second layer, and the first porous membrane independently has a height ranging from about 50 microns to about 4 mm, from about 100 microns to about 4 mm, from about 50 microns to about 2 mm, from about 100 microns to about 2 mm, from about 50 microns to about 1 mm, from about 100 microns to about 1 mm, from about 10 microns to 1 mm, from about 10 microns to about 4 mm, from about 10 microns to about 2 mm, from about 50 microns to about 500 microns, from about 100 microns to about 200 microns, from about 10 microns to 100 microns, or from about 15 microns to about 50 microns, and optionally wherein the first layer, the second layer, and the first porous membrane have the same height or have at least two different heights.
6. The microfluidic device of any one of paragraphs 1-5, wherein each of the first layer, the second layer, and the first porous membrane is independently formed of an inert, transparent material, optionally wherein the transparent material is polydimethylsiloxane, polyester, acrylic, nylon, polycarbonate, polylactic acid, polyethylene terephthalate glycol, polyether ether ketone, polyether ketone ketone, polypropylene, or glass, or a combination thereof, optionally wherein the first layer, the second layer, and the first porous membrane are formed of the same material or are formed of at least two different materials.
7. The microfluidic device of any one of paragraphs 1-6, wherein the shape of the first and second channels in the material exchange area is independently wavy or substantially straight,
   optionally wherein the shape of the first channel in the material exchange area is substantially straight, and wherein the shape of the second channel in the material exchange area is wavy;
   optionally wherein the shape of the first channel in the material exchange area is wavy and wherein the shape of the second channel in the material exchange area is substantially straight;
   optionally wherein the shape of both the first and second channels in the material exchange area is wavy; and
   optionally wherein the wavy shape is serpentine, sinewave, or triangle-wave, or a combination thereof.
8. The microfluidic device of any one of paragraphs 1-7, wherein the first or second layer further comprises a third channel, and wherein within the material exchange area, the third channel comprises a fourth region that overlaps with the first region of the first channel or the second region of the second channel, and/or overlaps with a fifth region of the second or first channel, and wherein the third channel comprises a sixth region that does not overlap with any region of the second or first channel in the material exchange area.
9. The microfluidic device of paragraph 8, wherein the third channel is in the first layer, and wherein within the material exchange area, the fourth region of the third channel of the first layer overlaps with the second region and/or the fifth region of the second channel of the second layer; or
   wherein the third channel is in the second layer, and within the material exchange area, the fourth region of the third channel of the second layer overlaps with the first region and/or the fifth region of the first channel of the first layer.
10. The microfluidic device of paragraph 8 or 9, wherein the third channel comprises a third fluid inlet within the feed area, and a third fluid outlet within the exit area, wherein the third fluid inlet and outlet are configured such that fluid flows in a third direction through the third channel, and wherein the third direction is the same as the first and second directions, or is different from the first and/or second direction(s).
11. The microfluidic device of any one of paragraphs 8-10, wherein the third channel is in the first layer, and wherein the shape of the third channel in the material exchange area is substantially straight or wavy.
12. The microfluidic device of any one of paragraphs 8-10, wherein the third channel is in the second layer, and wherein the shape of the third channel in the material exchange area is substantially straight or wavy.
13. The microfluidic device of any one of paragraphs 1-12, further comprising a third layer and a second porous membrane, wherein the third layer comprises a fourth channel, wherein the third layer is positioned on top of the second layer and in parallel with the first and second layers, wherein the second porous membrane is adjacent to each of and between the third and second layers, and wherein within the material exchange area, the fourth channel comprises a seventh region that overlaps with the second region, the fifth region, and/or an eighth region of the second channel, and wherein the fourth channel comprises a ninth region that does not overlap with any region of the second channel in the material exchange area.
14. The microfluidic device of paragraph 13, wherein the second porous membrane comprises a plurality of pores, and optionally wherein the pores in the plurality of pores have the same average diameter or comprise two or more groups, wherein each group of pores has a different average diameter.
15. The microfluidic device of paragraph 13 or 14, wherein each of the third layer and second porous membrane has a height ranging from about 50 microns to about 500 microns, from about 100 microns to about 200 microns, from about 10 microns to 100 microns, or from about 15 microns to about 50 microns, and optionally wherein the third layer and second porous membrane has the same height or two different heights.
16. The microfluidic device of any one of paragraphs 13-15, wherein the shape of the fourth channel in the material exchange area is substantially straight or wavy.
17. The microfluidic device of any one of paragraphs 1-16, wherein each layer of the microfluidic device comprises two or more channels.
18. The microfluidic device of any one of paragraphs 1-17, further comprising one or more reservoirs in fluid connection with at least one channel, optionally wherein the one or more reservoirs are fluidly connected to the floor of the at least one channel.
19. A microfluidic device comprising:
   at least two layers, each layer comprising two or more channels; and
   a first porous membrane,
   wherein the first layer is in parallel with the second layer,
   wherein the first porous membrane is adjacent to each of and between the first and second layers,
   wherein the microfluidic device comprises a material exchange area,
   wherein within the material exchange area, a first region of each channel in the first layer overlaps with a second region of at least one channel in the second layer, and
   wherein a third region of each channel in the first layer does not overlap with any region of any channel in the second layer in the material exchange area.
20. A method for constructing a disease model using the microfluidic device of any one of paragraphs 1-19, comprising:
   (i) placing a first type of cells in the first or second channel, optionally a second type of cells in the first and/or second channel; and
   (ii) flowing a first cell culture medium through the first or second channel having the first type of cells placed therein, optionally a second cell culture medium through the first and/or second channel.
21. The method of paragraph 20, wherein step (ii) is performed at about 37° C. for a time period ranging from 3 to 21 days or from 7 to 18 days.
22. The method of paragraph 20 or 21, wherein in step (ii), the first cell culture medium, and optionally the second cell culture medium, has an adequate flow rate such that normalized diffusion of 10% or less of the first cell culture medium diffuses through the first porous membrane into the second channel, optionally normalized concentration of 10% or less of the second cell culture medium diffuses through the first porous membrane into the first channel.
23. The method of any one of paragraphs 20-22, wherein secreted solutes by cells in one or more of the channels are transported to cells in a neighboring channel or neighboring channels via flow convection or diffusion.
24. The method of any one of paragraphs 20-23, further comprising, prior to step (i), coating the first channel with a first matrix and coating the second channel with a second matrix.
25. The method of any one of paragraphs 20-24, wherein at time points of 1 day, 2 days, 3, days, 4 days or 5 days following step (ii), at least two different types of tissues are formed in the channels of the microfluidic device, wherein at least 90% of at least one type of tissue is in the first channel, optionally at least 90% of at least one type of tissue is in the second channel.
26. The method of paragraph 25, wherein a first type of tissue is predominantly in the first channel and optionally a second type of tissue is predominantly in the second channel, and wherein one or more cells of the first type of tissue are in the second channel and/or one or more cells of the second type of tissue are in the first channel.
27. The method of any one of paragraphs 20-26, wherein when the microfluidic device comprises the third channel, the method further comprises in step (i) placing a third type of cells in the third channel and in step (ii) flowing a third cell culture medium through the third channel.
28. The method of paragraph 27, wherein in step (ii), the third cell culture medium has an adequate flow rate such that normalized concentration of 10% or less of the third cell culture medium diffuses through the first porous membrane into the first or second channel.
29. The method of any one of paragraphs 20-28, wherein when the microfluidic device comprises a third layer and a second porous membrane, the method further comprises in step (i) placing a fourth type of cells in the fourth channel and in step (ii) flowing a fourth cell culture medium through the fourth channel.
30. The method of paragraph 29, wherein in step (ii), the fourth cell culture medium has an adequate flow rate such that normalized concentration of 10% or less of the fourth cell culture medium diffuses through the second porous membrane into the second channel.
31. The method of any one of paragraphs 27-30, wherein following step (ii), at least three or at least four different types of tissues are formed in the microfluidic device.
32. The method of any one of paragraphs 20-31, wherein in step (i), the cells is placed in the form of a monolayer, an organoid, or a spheroid, or the cells being placed are embedded in a hydrogel or ex vivo tissue.
33. A method for drug screening using the microfluidic device of any one of paragraphs 1-19, comprising:
   (i) placing one or more type(s) of cells in the first channel and optionally one or more type(s) of cells in the second channel, wherein when one or more type(s) of cells are placed in the second channel, at least one type of cells in the second channel is different from the one or more type(s) of cells placed in the first channel;
   (ii) flowing a first cell culture medium through the first channel and optionally a second cell culture medium through the second channel to form at least two different tissue types; and
   (iii) flowing a drug solution or suspension or a drug carrier medium through the first channel and/or the second channel; and optionally
   (iv) flowing a fifth cell culture medium through the first channel and/or the second channel.
34. The method of paragraph 33, wherein when the microfluidic device comprises two or more channels in each layer, the method comprises
   in step (i) the one or more type(s) of cells are placed in all channels in the first layer, and optionally the one or more type(s) of cells are placed in all channels in the second layer;
   in step (ii) the first cell culture medium flows through all channels in the first layer, and optionally the second cell culture medium flows through all channels in the second layer; and
   in step (iii) a first drug solution or suspension, or a first drug carrier medium flows through one mor more channel(s) in the first layer, and/or a second drug solution or suspension, or a second drug carrier medium flows through one or more channel(s) in the second layer, wherein the first and second drug solution or suspension are the same or different.
35. The method of paragraphs 34, wherein when the microfluidic device comprises a third layer and a second porous membrane, the method further comprises optionally in step (i) placing a fourth type of cells in the third channel, optionally in step (ii) flowing a fourth cell culture medium through the third channel, and in step (iii) flowing a drug solution or suspension through the third channel.
36. The method of any one of paragraphs 33-35, wherein following step (ii), at least two or at least three different types of tissues are formed in the channels of the microfluidic device.
37. The method of any one of paragraphs 33-36, wherein in step (iii), the drug solution or suspension has an adequate flow rate and/or concentration such that a concentration gradient is produced along the first and/or second channels, and optionally along the fourth channel.
38. The method of any one of paragraphs 33-38, further comprising during or after step (iii), measuring cell responses to the drug.
39. An organ-on-a-chip platform comprising:
   one or more the microfluidic device of any one of paragraphs 1-19, and
   one or more types of tissue predominantly in the first channel and optionally one or more types of tissue predominantly in the second channel,
   wherein when two or more microfluidic devices are included, at least one channel of each microfluidic device is in fluid connection with another microfluidic device in the platform.
40. The platform of paragraph 39, wherein a first type of tissue is predominantly in the first channel and optionally a second type of tissue is predominantly in the second channel, and optionally wherein one or more cells of the first type of tissue are in the second channel and/or one or more cells of the second type of tissue are in the first channel.
41. The platform of paragraph 40, wherein when one or more microfluidic device(s) comprise(s) the third channel, the platform further comprises a third tissue predominantly in the third channel, optionally wherein one or more cells of the third tissue are in the second or first channel.
42. The platform of paragraph 40 or 41, wherein when one or more microfluidic device(s) comprise(s) the third layer and second porous membrane, the platform further comprises a fourth tissue predominantly in the fourth channel, optionally wherein one or more cells of the fourth tissue are in the second channel.

The disclosed devices and methods can be further understood through the following non-limiting examples and exemplary embodiments.

### EXAMPLES

### Example 1: Exemplary Bio-Engineered Human Prostate Disease Model

### Material and Methods

### Chip Design and Fabrication

An exemplary device includes two channels separated by a porous membrane, and inlet and outlet holes for connection with external flow operating system. One of the two channels is a straight channel having the dimension as described in Jiang, et al., Biomicrofluidics, 13, 064116 2019, and the other one is a 59 mm-long serpentine channel, 1 mm wide and 500 μm high. The serpentine channel consists of 16 orthogonal channel segments, perpendicular to the main channel axis, extending in both directions from the centerline axis of the straight channel to distal locations at a distance of 2.5 mm. The membrane sandwiched between the two channels has a pore size of 0.8 μm or 8 μm, selected to conduct a designed experiment. FIGS. 1a-1c and 2a-2b illustrate the device design CAD drawings and the device assembly.

The exemplary device is made of optically transparent polydimethylsiloxane (PDMS, Sylgard 184, Dow Corning Corporation) utilizing soft lithography technology. Two molds for the microchannels were fabricated in an aluminum block or using 3D printing technique, respectively, for the straight channel and the serpentine channel. PDMS mixture, prepared by mixing PDMS base and curing agent at a 10:1 w/w ratio, was dispensed on the mold. After removing air bubbles in the PDMS mixture under vacuum, the molds with the PDMS were placed on a leveled surface at room temperature until the PDMS was fully cured. The PDMS layer was peeled off from the mold to obtain microchannel replicates. Porous membrane, upon 1-minute treatment in an oxygen-plasma (Harrick Plasma, USA), was immediately immerged in 5% 3-aminopropyltriethoxysilane (APTES, Sigma-Aldrich) solution in water, and was incubated at 50° C. for about 30 min to prepare its surfaces for bonding. The bonding surfaces of a pair of PDMS channel replicates, with inlet and outlet holes, were activated by oxygen plasma. The pair of the PDMS channels, and the dried membrane, were then aligned and brought into firm contact. The assembled device was incubated at 50° C. for 30 min to establish strong bonding. Finally, inlet and outlet adapter tubing were assembled to the channel inlet and outlet holes for connections with the external flow control system.

As shown in FIGS. 1a-1c and 2a-2b, the design allows independent supply of the medium-constituents at desired flow rate to the two channels to maintain two cell cultures, one in each channel. In addition, the communications and interaction between the two cultures are realized through diffusion of the two media through the pores. Furthermore, drug can be administered at the inlet of one or both channels for treatment tests.

### Coatings

Before any coating, a channel was first washed by flowing 70% ETOH several times and incubated under UV irradiation inside the biosafety hood for sterilization. For example, after washing the channel with 1×PBS multiple times, a coating solution was introduced into the channel. The device was placed inside the biosafety hood under UV for a one-hour incubation at room temperature. The coating solution was then removed from the channel leaving a thin layer coating on the surfaces of the channel. The device was then placed inside the incubator to cure the coating for one hour at 37° C. A different coating in the other channel was conducted in a similar manner subsequently. After the coated layer was cured, the channel was washed with 1×PBS, and subsequently, washed with an appropriate medium, and incubated for more than 30 min before placing the cells inside the channel.

To provide good adhesion and physiological relevant matrix for the stroma cells on the membrane, the stroma channel was coated using 20 μg/ml fibronection (Sigma F0895) or a matrix of collagen/fibronectin/PEG, whereas the tumor channel was coated with 10 μg/ml laminin (original concentration 1.2 mg/ml). Fibronectin coating solution was diluted in 1×PBS w/o Ca/Mg to a concentration of 20 μg/ml, whereas laminin coating solution was diluted in 1×PBS w/Ca/Mg to obtain a concentration of 10 μg/ml. The solution of the matrix coating for stroma is a mixture of 500 μg/ml rat tail collagen (Corning 354236), 20 μg/ml fibronectin (Sigma F0895), and 62.5 μg/ml PEG in 1×PBS w/o Ca/Mg at pH 7.0 (adjusted by adding a couple microliter of 0.1M NaOH once at a time incrementally).

### Cell Cultures and Treatments

Immortalized primary human prostate basal epithelial cells (iPrECs) were used as the normal epithelium in the prostate grand model. PrECs were grown in keratinocyte serum-free medium (KSFM, Gibco 17005042) supplemented with bovine pituitary extract (BPE), epidermal growth factor (EGF), and 1% penicillin-streptomycin. Benign human prostate stromal cells (BHPrS1s) from Simon Hayward were used as the normal stroma. BHPrS1 cells were grown in RPMI 1640 medium (Gibco) with 5% fetal bovine serum (FBS) and 1% penicillin-streptomycin. Three types of tumor cell lines, EMP, C4-2 and 22 RV1 (ATCC), were used. EMP cells were generated by stably introducing Myc (M) shPten (P) and ΔErg (E) into PrEC cells. They were maintained in Keratinocyte Serum-Free Medium (KSFM, Gibco 17005042) supplemented with bovine pituitary extract (BPE), epidermal growth factor (EGF), and 1% penicillin-streptomycin. C4-2 and 22Rv1 cells were obtained from ATCC and grown in RPMI 1640 medium (Gibco) with 5% FBS, 1% penicillin-streptomycin. Human prostate cancer spheroids isolated from a patient were used in one experiment. The deidentified tissue was obtained from the tissue repository under a standard IRB protocol. For invasion experiments, fluorescently labeled cells were obtained by lentiviral infection with plasmids containing either green fluorescent protein (GFP) or red fluorescence protein (RFP) cDNAs. Specifically, mCherry-labeled BHPrS1 cells, GFP-labeled tumor cells, either C4-2 or 22Rv1, and normal PrEC cells were utilized. For observation of the tumor invasion, tumor cells (C4-2 and 22RV1) with color-reporters were used for co-culture experiments with color or non-color BHPrS1 cells. For control experiments of the invasion, co-culture experiments of GFP-labeled iPrECs with non-labeled BHPrS1 cells, as well as non-labeled iPrECs with mcherry color BHPrS1 cells, were conducted. All cultures were maintained in an incubator at 37° C. with 5% CO₂ with humidity >90%. Co-culture experiments lasted 7-18 days.

Synthetic androgen (R1881) was obtained from Perkin Elmer. For all co-culture experiments, i.e., PrEC/BHPrS1, C4-2/BHPrS1 and 22RV1/BHPrS1, independent medium flows at 30 μl/hr were provided for each cell type through the experiment with the R1881 treatment in the stromal medium only. R1881 was administered in the stroma medium through an in-flow adapter every two days at average concentration of 50 nM.

Drug treatment experiments were performed in devices. HYD-1 was administered to the tumor medium supply at a concentration in a range of 14 μM-50 μM, on the alternate dates to the R1881 treatment.

### Immunofluorescence

Antibodies ITGα6 (GoH3) (sc-19622), AR (sc-7305), and p63 (sc-56188) and POSTIN (sc-398631) were purchased from Santa Cruz. TMPRSS2 was obtained from University of Washington. HMWCK was obtained from Dako. Vimentin (V4630), KRT19 (HPA002465), and αSMA (A2547) were obtained from Millipore/Sigma. CoL1A1 (Gene Tex GTX112731) and FAP (Gene Tex GTX102732) were obtained from Gene Tex. Keratin 19 (ab76539) was obtained from Abcam.

The immune-staining processes were performed by flowing the solutions through the channels with the cells on each side of the membrane inside the channels. The cells were first washed several times using 1×PBS, they were then fixed with 4% paraformaldehyde at room temperature for about 10 min. Permeabilization was carried out using 0.2% Triton for 6 min, followed by 4 washes with 1×PBS. Goat serum (5%), donkey serum (5%) or BSA (1%), was used for 1 h blocking at room temperature. Cells were then incubated in designated primary antibody solutions diluted in the blocking solutions at 4° C. overnight. After 4 washes with 1×PBS, secondary antibody solutions were flowed into the channels for 1 h-incubation at room temperature. The cells were washed thoroughly using 1×PBS and followed, when needed, by nucleus staining with 10 μg/ml Hoescht (Sigma-Aldrich) for 10 min. Fluorescence imaging were conducted using a Nikon Eclipse TE2000-U microscope under various objectives (4×, 10×, 20×). For taking images under higher magnifications (40×, and 100×), the devices were opened to obtained the membrane with cells. The membrane was mounted in 70%-90% glycerol solution and was sandwiched between two #1.5 glass coverslips.

### Results and Discussion

### Flow Simulations

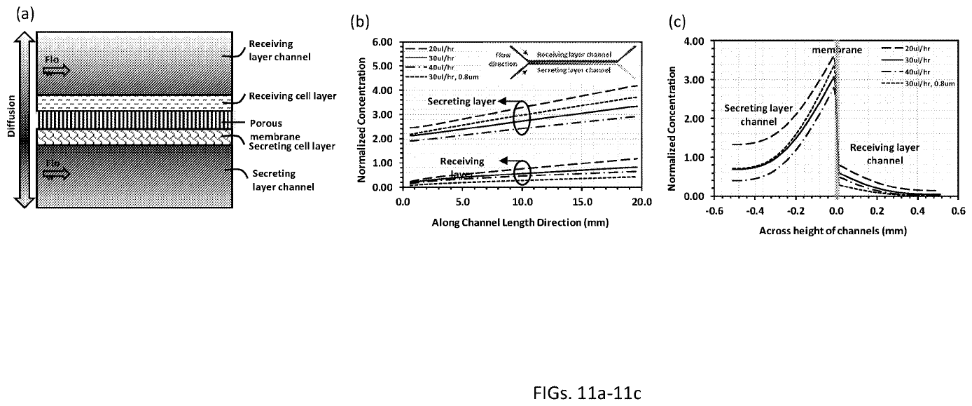

Numerical computations were conducted using COMSOL to gain insight into molecules transport interaction in the co-culture. A simplified 2D physical model featuring two identical straight channels aligned and separated by porous membrane was adopted for the simulations to highlight the convective-diffusive transport of signalling molecules (FIG. 11a). Each straight channel was 20 mm long and 500 μm high, while the pore size of the 20 μm-thick separation membrane was 8 μm with 2% porosity. The communication between the 2 cell layers in the co-culture is bi-directional; namely, each cell layer is simultaneously secreting and receiving signals. However, for illustration purposes, a cell culture on one membrane surface was selected as the secreting layer with the other cell culture on the opposite membrane surface as the receiving layer; physical thicknesses of both cell layers were considered zero in the model. The effect of PDMS absorption of molecules was negligible under steady state.

Streamwise normalized concentration profiles of secreted molecules along the channels, from inlet to outlet at a distance 40 μm away from both membrane surfaces, were presented for 20, 30, and 40 μL/h flow rates (FIG. 11b). All concentrations monotonically increased along the flow direction attributed to the balance between stream-wise convection and cross-stream diffusion of secreted molecules. Normalized concentration profiles of the receiving cell layer showed similar trends, i.e., increasing linearly along the flow direction, showing that the receiving layer microenvironment near the channel outlet contained significantly higher concentration of the secreted molecules than the microenvironment near the channel inlet. Furthermore, at the same stream-wise location, the concentration in the receiving channel was systematically about 20% of the secreting channel concentration. Increasing flow rate led to reduced concentrations in both secreting and receiving channels. Cross-stream normalized concentration profiles, at half distance between the channels inlets and outlets, are presented for the same flow rates (FIG. 11c). The results showed strong concentration gradients across the cell layers on each side of the membrane, which may be due to the low effective diffusion coefficient. In the receiving layer channel, concentration was high at the cell layer on the membrane, but rapidly diminished at locations father away from the cell layer, showing that signalling molecules were well confined to the vicinity of the receiving cell layer. Similarly, in the secreting channel, the concentrations decreased from their peaks at the secreting cell layer with increasing distance away from the membrane. The simulation was repeated for the 0.8 μm-pore membrane device with a lower diffusion (porosity 0.8%). The results (dotted curves in FIGS. 11b and 11c) showed similar concentration profiles in both streamwise and cross-stream directions with enhanced gradients across the cell layers. The diminishing concentration away from the receiving cell layer indicated a limited diffusion range at a reduced concentration level. Similarly, drug concentration distributions under different schemes can be obtained by selecting adequate t flow rates, porosity, and drug administering conditions (dosage, frequency, times, in a single-channel or in both channels, etc.).

### Co-Culture of Tumor Cells With Normal Human Prostate Fibroblasts and Cancer-Associated Fibroblasts (CAF) Conversion

To investigate the influence of tumor cells on the stroma within the devices, prostate tumor cells were co-cultured with normal prostate fibroblasts in disease model described in FIGS. 9a-9d. Co-culturing normal stroma with an oncogene-induced human tumor cell line EMP or with metastatic C4-2 or 22 RV1 tumor cells in either 0.8 um or 8 um pore devices induced the expression of CAF markers αSMA and Col1A1 in the stroma. This shows that secretive solute from the tumor cells can convert stroma into CAF without physical contact.

Tumor cells induced Col1A1 and αSMA in distinct stromal populations, showing heterogeneity within the CAF population. Moreover, co-culture of primary prostate tumor from a patient with the normal stroma also converted the stroma to CAFs where αSMA and Col1A1 were expressed in distinct subsets of stromal cells. In contrast, Col1A1 and αSMA were nearly undetectable in the stroma co-cultured with normal prostate epithelial cells (PrECs). Tumor cells also induced loss of Androgen Receptor (AR) expression in stroma in co-culture in the devices. AR expression, which was very high and localized in stroma cell nucleus when co-cultured with normal PrECs, was significantly reduced in the stroma co-cultured with tumorigenic EMP cells or C4-2 cancer cells.

### Tumor Cells Invasion Into Stroma Channel

Prostate tumor cells (GFP-tagged 22RV1 or C4-2) co-cultured with normal human stromal fibroblasts (mCherry-tagged BHPrS) in 8 μm-pore devices invaded into the stroma channel through the pores of the membrane at the overlapping channel regimes. The invaded tumor cells, adhering to the BHPrS cells, could not be removed by flow. BHPrS cells, converted into CAFs expressing αSMA, Col1A1, POSTIN and FAP, were also observed in the tumor cell channels. Collected cells from each of the BHPrS and tumor cell channels both contained mixtures of BHP and tumor cells. Upon placed into culture wells, CAF fibroblasts spread simultaneously and developed into a full layer on the well surface within a couple of days, while tumor cells suspended in the culture medium initially, and seeded on the well surface in several days. Both cells were proliferating.

In another experiment, stromal fibroblasts and tumor cells were placed in direct contact in the disease model as illustrated in FIGS. 10a-10d. Normal BHPrS1 fibroblasts were plated on the membrane in the lower straight channel to obtain a confluent monolayer. Subsequently, GFP-tagged C4-2 cancer cells were placed directly on top of the fibroblast cell layer establishing 2 cell layers in direct physical contact in the same lower channel. The upper serpentine channel was left empty without cells. Culture medium flow was driven through each of the upper and lower channels at a constant flow rate of 30 μL/h, and R1881 treatment was administered only in the lower channel containing both cell types. Stromal fibroblasts were first observed in the upper channel on Day 2 of the co-culture, migrating from the overlapping regions of the straight seeding channel into the upper serpentine channel. In the ensuing few days, the stromal fibroblasts rapidly spread over the entire serpentine channel surface reaching all distal locations. In this configuration, the C4-2 cancer cells followed the stromal fibroblasts advancing into the serpentine channel at a very fast pace. Many C4-2 cancer cells were detected at the farthest distal locations in the serpentine channel about 2 mm away from their original straight channel by Day 12. They appeared. mostly as single cells or in small cell clusters with some noticeable individual cells, not rounded but spreading on the channel surface. The C4-2 cancer cells remaining in their original channel also changed morphologically from a monolayer into a disrupted 3D layer reaching the surface facing the separation membrane of the lower channel that had been covered with stromal fibroblasts. This could be attributed to both a high proliferation rate and improved adherence to the channel surface modified by the stromal fibroblasts. In comparison with experiments where stromal fibroblasts and cancer cells were plated on opposite sides of the membrane, the direct physical contact of the cancer cells with fibroblast dramatically increased the number of invading tumor cells along with their migration range and pace. Similar experiment was repeated with GFP-22Rv1 cancer cells placed directly on top of a confluent monolayer of mCherry-BHPrS1 fibroblasts in the lower straight channel, while the top serpentine channel was left empty without cells. By Day 3, mCherry-fibroblasts were first observed entering the orthogonal channel segments of the serpentine channel, followed by several GFP-22Rv1 cancer cells. In this case, 22Rv1 cells reached the farthest distal location of the serpentine channel by Day 7, exhibiting enhanced invasion and spreading capability in comparison with 22Rv1 co-cultured with BHPrS1 cell on opposite sides of the membrane without direct physical contact.

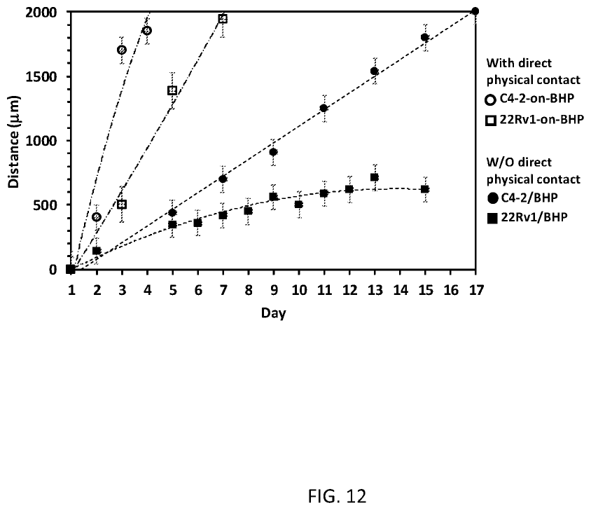

The disease models (FIGS. 9a-9d and 10a-10d) allow quantitative assessment of the cancer cell migration rate under different initial conditions. 22Rv1 and C4-2 cells exhibited migration rate of 0.04 μm/min and 0.08 μm/min, respectively, in the no-physical-contact model, both showed increased rates, 0.2 μm/min or 0.4 μm/min, respectively, in the physical-contact disease model (FIG. 12).

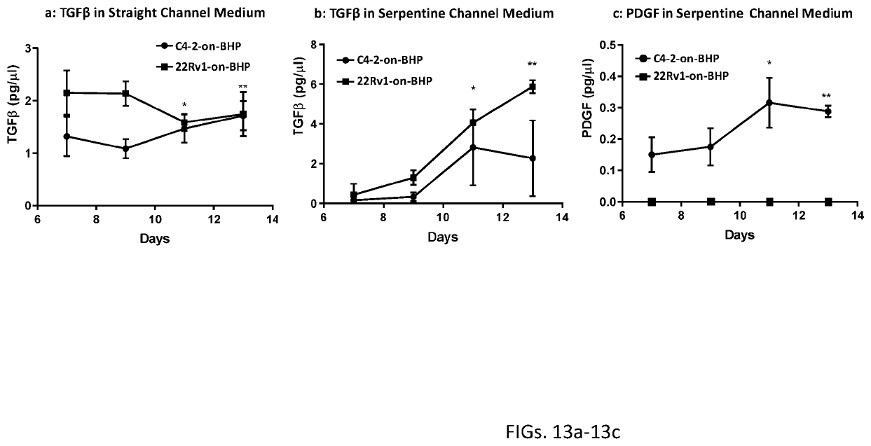

In addition, secretion of cytokines, TGFβ, PDGF, and PDGFR, by the tumor cells were evaluated using conditioned medium samples collected from experiments with physical contact between tumor and stroma cultures (FIGS. 10a-10d). Samples were collected separately from tumor and stroma channels at 2-day intervals from Day 7 to Day 13 of the co-culture, and analyzed using ELISA. The production of TGFβ in straight channel medium (initial tumor cell/stroma plating channel) was robustly detected. Higher TGFβ levels were initially produced in “22Rv1 on BHP”, but slightly dropped later, while the levels in “C4-2 on BHP” slightly increased with time (FIG. 13a). In the serpentine channel, where most of the cells were CAFs, the level of TGFβ in its medium increased with time for the two co-cultures (FIG. 13b). This could be attributed to continuous increase in the number of CAFs, over the tested period, producing higher levels of TGFβ. In addition, the number of tumor cells in the serpentine channel also increased over time, from newly invaded tumor cells and cell division of the early invaded tumor cells. Low levels of PDGF were detected only in the medium collected from the serpentine channel of the “C4-2 on BHPrS1” co-culture, increasing with time presumably due to the observed higher rates in migration and proliferation of the invaded C4-2 cells and CAFs (FIG. 13c). PDGF was below detectable levels in the medium collected from the serpentine channel of the “22Rv1 on BHP” and those from the straight channels of both experiments. TNFα was not detectable in any samples of both experiments, consistent with the absence of immune cells in this model.

### Response to Drug Treatment

Drug tests were also investigated under various conditions by varying the treatment dosage, treatment frequency, treatment total times, etc. Roles of stroma on the treatment of tumor cells were also investigated by varying the co-culture arrangement, e.g., tumor cells and stroma in different channels on each side of the pore membrane, or tumor cells placed on top of a full layer of stroma in the same channel. Drug treatment was administered either in one of the channels or in both channels.

For example, tumor cells (C4-2) were under treatment of HYD-1 in the disease model without initial direct physical contact between tumor cells and stroma (FIGS. 9a-9d). The treatment was provided at low concentrations in a range of 10-20 μM HYD-1 in medium flow only to the tumor cell channel, while culture medium without drug was flowed into the stroma channel. At the initial two days of the treatment, tumor cells did not stop invading into the stroma channel. With increasing concentration, and/or increasing treatment times, tumor cells became dead after 4 days. A clear distribution of tumor cell color reporter intensity along the channel was observed, where cells lost green signal, indicating their death, at the drug-containing medium flow inlet but alive in bright green color at the flow outlet. In addition, tumor cells exhibiting bright green signal remained at the overlapping regimes of the two channels, where interactions between tumor cells and stromal fibroblasts were present. The result demonstrated that stroma played a role in tumor cell survival and resistance to drug treatment. In another experiment using the same disease model (FIG. 9), drug HYD-1 containing culture medium was flowed into both channels of tumor and stroma. Death of tumor cells were observed along the channel including the overlapping regimes. The initially fully confluent stromal fibroblasts became deteriorating and detached from the channel surface. This showed that treatment to both tumor cells and the surrounding stroma in the tumor microenvironment was more effective. Drug treatment was also tested in another disease model, where tumor cells and stroma were initially placed with direct physical contact in the same channel (FIGS. 10a-10d). Drug of 50 μM HYD-1 in culture medium was flowed into the tumor/stroma channel while culture medium flow was supplied to the second empty channel. After one-day treatment, gradient in green signal (tumor cell live/death reporter) along the channel from flow inlet to outlet was observed. An additional 3-day of treatment resulted in more dead tumor cells in the channel while only some tumor cells were alive downstream to the flow outlet. After treatment of 8 days, most tumor cells were dead along with deteriorated stroma. This result further confirmed the role of stroma on effectiveness of drug treatment in tumor cells.

It is understood that the disclosed method and compositions are not limited to the particular methodology, protocols, and reagents described as these can vary. It is also to be understood that the terminology used herein is for the purpose of describing particular forms only, and is not intended to limit the scope of the present invention which will be limited only by the appended claims.

It must be noted that as used herein and in the appended claims, the singular forms “a” , “an”, and “the” include plural reference unless the context clearly dictates otherwise. “Optional” or “optionally” means that the subsequently described event, circumstance, or material may or may not occur or be present, and that the description includes instances where the event, circumstance, or material occurs or is present and instances where it does not occur or is not present.

Those skilled in the art will recognize, or be able to ascertain using no more than routine experimentation, many equivalents to the specific forms of the method and compositions described herein. Such equivalents are intended to be encompassed by the following claims.

1. A microfluidic device comprising:
at least two layers, each layer comprising a channel; and
a first porous membrane,
wherein the first layer is in parallel with the second layer,
wherein the first porous membrane is adjacent to each of and between the first and second layers,
wherein the microfluidic device comprises a material exchange area,
wherein within the material exchange area, a first region of a first channel overlaps with a second region of a second channel, and
wherein a third region of the first channel does not overlap with any region of the second channel in the material exchange area.

2. The microfluidic device of claim 1, wherein the microfluidic device comprises a feed area and an exit area, wherein within the feed area, each channel comprises a fluid inlet, and within the exit area, each channel comprises a fluid outlet, wherein each of the fluid inlets and fluid outlets are configured such that fluid flows in a first direction through the first channel and fluid flows in a second direction through the second channel, and wherein the first direction and the second direction are the same or different, optionally wherein the first direction is the same as the second direction.

3. The microfluidic device of claim 1, wherein the first porous membrane comprises a plurality of pores, and optionally wherein the pores in the plurality of pores have the same average diameter or comprise two or more groups, where each group of pores has a different average diameter.

4. The microfluidic device of claim 3, wherein the plurality of pores have an average diameter ranging from about 0.4 micron to about 15 microns, from about 0.4 micron to about 10 microns, from about 0.4 micron to about 5 microns, from about 0.4 micron to about 1 microns, from about 0.8 micron to about 15 microns, from about 1 micron to about 15 microns, from about 0.8 micron to about 10 microns, from about 1 micron to about 10 microns, from about 0.8 micron to about 8 microns, from about 0.8 micron to about 5 microns, from about 2 micron to about 15 microns, or from about 5 micron to about 15 microns; or
wherein the plurality of pores comprises two or more groups, wherein a first group of pores has a first average diameter ranging from about 0.4 micron to 5 micron, and a second group of pores has a second average diameter ranging from 0.8 micron to about 15 microns, and wherein the first average diameter is different from the second average diameter.

5. The microfluidic device of claim 1, wherein each of the first layer, the second layer, and the first porous membrane independently has a height ranging from about 50 microns to about 4 mm, from about 100 microns to about 4 mm, from about 50 microns to about 2 mm, from about 100 microns to about 2 mm, from about 50 microns to about 1 mm, from about 100 microns to about 1 mm, from about 10 microns to 1 mm, from about 10 microns to about 4 mm, from about 10 microns to about 2 mm, from about 50 microns to about 500 microns, from about 100 microns to about 200 microns, from about 10 microns to 100 microns, or from about 15 microns to about 50 microns, and optionally wherein the first layer, the second layer, and the first porous membrane have the same height or have at least two different heights.

6. The microfluidic device of claim 1, wherein each of the first layer, the second layer, and the first porous membrane is independently formed of an inert, transparent material, optionally wherein the transparent material is polydimethylsiloxane, polyester, acrylic, nylon, polycarbonate, polylactic acid, polyethylene terephthalate glycol, polyether ether ketone, polyether ketone ketone, polypropylene, or glass, or a combination thereof, optionally wherein the first layer, the second layer, and the first porous membrane are formed of the same material or are formed of at least two different materials.

7. The microfluidic device of claim 1, wherein the shape of the first and second channels in the material exchange area is independently wavy or substantially straight,
optionally wherein the shape of the first channel in the material exchange area is substantially straight, and wherein the shape of the second channel in the material exchange area is wavy;
optionally wherein the shape of the first channel in the material exchange area is wavy and wherein the shape of the second channel in the material exchange area is substantially straight;
optionally wherein the shape of both the first and second channels in the material exchange area is wavy; and
optionally wherein the wavy shape is serpentine, sinewave, or triangle-wave, or a combination thereof.

8. The microfluidic device of claim 1, wherein the first or second layer further comprises a third channel, and wherein within the material exchange area, the third channel comprises a fourth region that overlaps with the first region of the first channel or the second region of the second channel, and/or overlaps with a fifth region of the second or first channel, and wherein the third channel comprises a sixth region that does not overlap with any region of the second or first channel in the material exchange area.

9. The microfluidic device of claim 8, wherein the third channel is in the first layer, and wherein within the material exchange area, the fourth region of the third channel of the first layer overlaps with the second region and/or the fifth region of the second channel of the second layer; or
wherein the third channel is in the second layer, and within the material exchange area, the fourth region of the third channel of the second layer overlaps with the first region and/or the fifth region of the first channel of the first layer.

10. The microfluidic device of claim 8, wherein the third channel comprises a third fluid inlet within the feed area, and a third fluid outlet within the exit area, wherein the third fluid inlet and outlet are configured such that fluid flows in a third direction through the third channel, and wherein the third direction is the same as the first and second directions, or is different from the first and/or second direction(s).

11. The microfluidic device of claim 8, wherein the third channel is in the first layer or in the second layer, and wherein the shape of the third channel in the material exchange area is substantially straight or wavy.

12. (canceled)

13. The microfluidic device of claim 1, further comprising a third layer and a second porous membrane, wherein the third layer comprises a fourth channel, wherein the third layer is positioned on top of the second layer and in parallel with the first and second layers, wherein the second porous membrane is adjacent to each of and between the third and second layers, and wherein within the material exchange area, the fourth channel comprises a seventh region that overlaps with the second region, the fifth region, and/or an eighth region of the second channel, and wherein the fourth channel comprises a ninth region that does not overlap with any region of the second channel in the material exchange area.

14. The microfluidic device of claim 13, wherein:
(i) the second porous membrane comprises a plurality of pores, and optionally wherein the pores in the plurality of pores have the same average diameter or comprise two or more groups, wherein each group of pores has a different average diameter;
(ii) each of the third layer and second porous membrane has a height ranging from about 50 microns to about 500 microns, from about 100 microns to about 200 microns, from about 10 microns to 100 microns, or from about 15 microns to about 50 microns, and optionally wherein the third layer and second porous membrane have the same height or two different heights; and/or
(iii) the shape of the fourth channel in the material exchange area is substantially straight or wavy.

15-16. (canceled)

17. The microfluidic device of claim 1, wherein each layer of the microfluidic device comprises two or more channels.

18. The microfluidic device of claim 1, further comprising one or more reservoirs fluid connected to at least one channel, optionally wherein the one or more reservoirs are fluidly connected to the floor of the at least one channel.

19. A microfluidic device comprising:
at least two layers, each layer comprising two or more channels; and
a first porous membrane,
wherein the first layer is in parallel with the second layer,
wherein the first porous membrane is adjacent to each of and between the first and second layers,
wherein the microfluidic device comprises a material exchange area,
wherein within the material exchange area, a first region of each channel in the first layer overlaps with a second region of at least one channel in the second layer, and
wherein a third region of each channel in the first layer does not overlap with any region of any channel in the second layer in the material exchange area.

20. A method for constructing a disease model using the microfluidic device of claim 1, comprising:
(i) placing a first type of cells in the first or second channel, optionally a second type of cells in the first and/or second channel; and
(ii) flowing a first cell culture medium through the first or second channel having the first type of cells placed therein, optionally a second cell culture medium through the first and/or second channel.

21-32. (canceled)

33. A method for drug screening using the microfluidic device of claim 1, comprising:
(i) placing one or more type(s) of cells in the first channel and optionally one or more type(s) of cells in the second channel, wherein when one or more type(s) of cells are placed in the second channel, at least one type of cells in the second channel is different from the one or more type(s) of cells placed in the first channel;
(ii) flowing a first cell culture medium through the first channel and optionally a second cell culture medium through the second channel to form at least two different tissue types; and
(iii) flowing a drug solution or suspension or a drug carrier medium through the first channel and/or the second channel; and optionally
(iv) flowing a fifth cell culture medium through the first channel and/or the second channel.

34-38. (canceled)

39. An organ-on-a-chip platform comprising:
one or more the microfluidic device of claim 1, and
one or more types of tissue predominantly in the first channel and optionally one or more types of tissue predominantly in the second channel,
wherein when two or more microfluidic devices are included, at least one channel of each microfluidic device is in fluid connection with another microfluidic device in the platform.

40. The platform of claim 39, wherein a first type of tissue is predominantly in the first channel and optionally a second type of tissue is predominantly in the second channel, and optionally wherein one or more cells of the first type of tissue are in the second channel and/or one or more cells of the second type of tissue are in the first channel, optionally, wherein:
(i) when one or more microfluidic device(s) comprise(s) a third channel, the platform further comprises a third tissue predominantly in the third channel, optionally wherein one or more cells of the third tissue are in the second or first channel; or
(ii) when one or more microfluidic device(s) comprise(s) a third layer and a second porous membrane, the platform further comprises a fourth tissue predominantly in a fourth channel, optionally wherein one or more cells of the fourth tissue are in the second channel.

41-42. (canceled)
